(19) United States
(12) Patent Application Publication
(10) Pub. No.: US 2024/0130768 A1
(43) Pub. Date: Apr. 25, 2024

(54) TAPERED ANCHORS

(71) Applicant: OrthoPediatrics Corp., Warsaw, IN (US)

(72) Inventors: Lawrence Haber, New Orleans, LA (US); Rick Detlefsen, Warsaw, IN (US); Matthew Prygoski, North Liberty, IN (US); Evangelos Tozakoglou, Fort Wayne, IN (US)

(73) Assignee: OrthoPediatrics Corp., Warsaw, IN (US)

(21) Appl. No.: 18/334,920

(22) Filed: Jun. 13, 2023

Related U.S. Application Data
(63) Continuation of application No. 16/657,252, filed on Oct. 18, 2019, now abandoned.
(60) Provisional application No. 62/747,764, filed on Oct. 19, 2018.
(60) Provisional application No. 62/750,500, filed on Oct. 25, 2018.

(51) Int. Cl. A61B 17/86; A61B 17/70

(52) U.S. Cl. CPC A61B 17/8625; A61B 17/7053; A61B 2017/564; A61B 2017/8655

(57) ABSTRACT

Bone anchors providing tensioning between adjacent bones, including a vertebra. Various embodiments use combinations of bone fasteners and staples to attach a tethering head to a bone. In some embodiments, the tether is attached to the head by means of a separate ring. In still other embodiments the tension in the tether is adjusted by use of a variable depth pocket within the head and/or the bone anchor.

### CROSS REFERENCE TO RELATED APPLICATION

This application is a continuation of U.S. patent application Ser. No. 16/657,252, filed Oct. 18, 2019, which claims the benefit of priority to U.S. Provisional Patent Application Ser. No. 62/747,764, filed Oct. 19, 2018, entitled TAPERED ANCHORS FOR SPINAL TETHERS, and to U.S. Provisional Patent Application Ser. No. 62/750,500, filed Oct. 25, 2018, entitled TAPERED ANCHORS WITH PUSHROD, all of which are incorporated herein by reference.

### FIELD OF THE INVENTION

Various embodiments herein pertain to bone anchors, and in particular for bone anchors adapted and configured for receiving a tension load from an adjacent bone anchor.

### SUMMARY OF THE INVENTION

Various embodiments described herein pertain to bone anchors that are interconnected in tension by one or more flexible members.

Preferably, the flexible member is of the type that can sustain a load in tension, but which is generally unsuitable for maintaining a compressive load. Various examples of the flexible members or connections include sutures, cables, springs, and tethers, including tape, woven tape, braided sutures, and flat braids, as various non-limiting examples. It is understood that some types of flexible connections (such as springs) may be capable of applying a limited range of moments from one connector to another connector.

In some embodiments, the anchor includes an internal cavity that is configured to receive within it a length of flexible member. In some embodiments the flexible member enters the internal cavity or pocket from an entrance, loops around the bottom of an adjustable member, and extends out of the pocket through an exit. It is understood that the terms entrance and exit are intended to represent different pathways for the flexible connector, and not necessarily with reference to the concepts of in or out.

Some aspects of the present invention include bone anchors having internal pockets that have adjustable depths. In some designs, the pocket includes internal threads that receive within them a set screw. Preferably, the pocket and the set screw extend distally within the shank of the fastener, and by adjustment of the set screw within the threaded pocket the distance from the entrance or exit to the underside of the set screw can be varied. This ability to vary the depth of the internal pocket provides the surgeon with means for adjusting the tension in the flexible member, once a sufficient portion of the slack of the flexible member is placed within the pocket and under the set screw.

In some embodiments, the bone anchor includes a chute or groove that extends downward from a respective entrance or exit, this chute being configured to slidingly receive within it the flexible member. Preferably, these distally and axially directed grooves extend through the threads of the internal pocket to provide for the smooth movement of the flexible member. However, the internal pocket retains sufficient threads to maintain engagement with the set screw.

In some embodiments, a separate member or pad is placed under the bottom of the set screw and over top of the innermost portion of the flexible member. Therefore, as the set screw is moved within the internal pocket, the set screw pushes down on the pad, and thereby places tension of the flexible member. In some embodiments, the pad includes alignment features that align the pad within the internal axial grooves.

Still further aspects of the present invention pertain to the use of a separate ring that is releaseably captured on the top of the head of the bone anchor. This ring includes a first mooring location at which an end of the flexible member is attached. The other end of the flexible member is preferably attached to a separable member that includes a second mooring location. In some instances, an anchoring body is sold with the flexible member connected to both the first and second mooring locations on one particular anchor. However, after implantation the ring can be removed and placed on an adjacent second anchor having a head that is configured to receive the ring.

In some embodiments, a separable ring is provided that attaches to a head of an anchoring assembly by means of a pivotal arm. The arm preferably has a free end that can latch with a corresponding feature of the head. In yet other embodiments, there is a separable ring that includes a spring that can be deflected out of the way during attachment to a head, but which moves back into an interlocking position after the interference is relieved.

Yet another aspect of the present invention pertains to a first separable ring that is maintained on the head by means of a second separate locking ring. When the locking ring is installed in grooves on both the head and the first separable ring, the second separable ring provides interference that prevents removal of the first separable ring from the head.

In yet another aspect of the present invention, the bone anchoring assembly includes a preferably smooth-walled internal pocket that receives within it a loop of flexible connector that extends from the entrance to the exit. The anchor preferably includes an adjustment screw with a central aperture that aligned with the internal pocket. A rod can be placed through the through aperture and into the pocket, so as to press down on the internal portion of flexible member, and thereby provide tension into the flexible member. After the tension is applied, the adjustment screw can be rotated (with the rod in place) so as to compress the flexible member between the head and internal surfaces of the set screw.

It will be appreciated that the various apparatus and methods described in this summary section, as well as elsewhere in this application, can be expressed as a large number of different combinations and subcombinations. All such useful, novel, and inventive combinations and subcombinations are contemplated herein, it being recognized that the explicit expression of each of these combinations is unnecessary.

### BRIEF DESCRIPTION OF THE DRAWINGS

Some of the figures shown herein may include dimensions. Further, the figures shown herein may have been created from scaled drawings, scaled models, or from photographs that are scalable. It is understood that such dimensions, or the relative scaling within a figure, are by way of example, and not to be construed as limiting unless so stated in a claim. Persons of ordinary skill will also recognize that CAD renderings may include lines that pertain to changes in surface geometry, and not necessarily to component features.

FIG. 1 is a top plan view of a CAD rendering of an anchor assembly according to one embodiment of the present invention.

FIG. 2 is an orthogonal, side elevational view of the apparatus of FIG. 1.

FIG. 3 is an orthogonal, bottom plan view of the apparatus of FIG. 2.

FIG. 4A is a cross sectional view of the apparatus of FIG. 2 for a cross section taken along the centerline of FIG. 2, and in the plane of FIG. 2.

FIG. 4B is a cross sectional view of the apparatus of FIG. 2 as taken along line 4B-4B.

FIG. 5A is a top plane view of a portion of the apparatus of FIG. 1.

FIG. 5B is an orthogonal, side elevational view of the apparatus of FIG. 5A.

FIG. 5C is an orthogonal, end elevational view of the apparatus of FIG. 5A.

FIG. 5D is a perspective view of the apparatus of FIG. 5A.

FIG. 6 is a top, exploded view of the apparatus of FIG. 1.

FIG. 7 is a perspective, cross sectional view similar to FIG. 4A, but with the arm rotated out of engagement.

FIG. 8 is a top perspective representation of a portion of the apparatus of FIG. 1.

FIG. 9 shows side-by-side a pair of the apparatus shown in FIG. 1.

FIG. 10 is a top plan view of a CAD rendering of an anchoring assembly according to another embodiment of the present invention.

FIG. 11A is a side elevational, orthogonal view of the apparatus of FIG. 10.

FIG. 11B is an orthogonal, end elevational view of the apparatus of FIG. 11A.

FIG. 12 is an orthogonal, bottom plan view of the apparatus of FIG. 11A.

FIG. 13A is an end elevational view of a portion of the apparatus of FIG. 10.

FIG. 13B is an orthogonal, top plan view of the apparatus of FIG. 13A.

FIG. 13C is an orthogonal, end elevational view of the apparatus of FIG. 13B.

FIG. 13D is an orthogonal, side elevational view of the apparatus of FIG. 13B.

FIG. 14 is a perspective, exploded view of the apparatus of FIG. 1.

FIG. 15 is a cross sectional view of the apparatus of FIG. 11A, taken down the centerline of FIG. 11A, and in the plane of the drawing.

FIG. 16 is a partial exploded view of the apparatus of FIG. 14.

FIG. 17 is a perspective view of the apparatus of FIG. 16, as taken from a different perspective.

FIG. 18 is a reassembled view of the apparatus of FIG. 16, with the ring rotated into its proper location.

FIG. 19 is a partially see-through representation of a portion of the apparatus of FIG. 18.

FIG. 20 is a top plan view of a CAD rendering of an anchoring assembly according to another embodiment of the present invention.

FIG. 21 is a side elevational, orthogonal view of the apparatus of FIG. 10.

FIG. 22 is an orthogonal, bottom plan view of the apparatus of FIG. 11A.

FIG. 23 is an orthogonal, end elevational view of the apparatus of FIG. 11A.

FIG. 24 is a cross sectional side elevational view of the apparatus of FIG. 11B, as taken along line 24-24.

FIG. 25 is a side elevational cross sectional view of the apparatus of FIG. 23, as taken down the centerline of the apparatus of FIG. 23, and parallel to the plane of the drawing.

FIG. 26 is a perspective exploded view of the apparatus of FIG. 20.

FIG. 27 is an enlarged view of a portion of the apparatus of FIG. 26, as taken from a different perspective.

FIG. 28 is a perspective representation of a pair of the apparatus shown in FIG. 20.

FIG. 29 is a side elevational view of a CAD rendering of a plurality of the apparatus of FIG. 20, with some of the anchors being modified.

FIG. 30 is a side elevational view of a CAD rendering of a plurality of the anchors of FIG. 20 used in conjunction with a pair of alternative anchoring assemblies.

FIG. 31 is a side elevational view of a CAD rendering of an anchor as shown in FIG. 20 used with a plurality of other anchoring assemblies.

FIG. 32 is a top plan view of a CAD rendering of an anchor according to another embodiment of the present invention.

FIG. 33 is a side elevational view of the anchor of FIG. 32, and presented orthogonally to FIG. 32.

FIG. 34 is a side elevational view of the apparatus of FIG. 33 from the opposite side.

FIG. 35 is an exploded view of the apparatus of FIG. 32.

FIG. 36 is a cutaway view of the apparatus of FIG. 32 as taken along line 36-36 of FIG. 32.

FIG. 37 is a perspective view of a CAD rendering of an anchor and an adjusting tool according to one embodiment of the present invention.

FIG. 38 is a side elevational view of a CAD rendering of a plurality of adjacent anchors as shown in FIG. 36.

FIG. 39 is a top plan view of a compression pad according to one embodiment of the present invention.

FIG. 40 is a side elevational view of a CAD rendering of an anchoring assembly according to another embodiment of the present invention.

FIG. 41 is a top, side perspective, semi-exploded view of the apparatus of FIG. 40.

### ELEMENT NUMBERING

The following is a list of element numbers used with all of the embodiments, and at least one noun used to describe that element. The “X” for all of these numbers is removed or replaced with a number (0 or greater) in the text and drawings of this application. Consistent with statements made elsewhere in this specification, these various 2-digit element numbers are used among multiple embodiments, and aspects of a particular element stated for one embodiment can be applied to the same element number in a different embodiment, except as shown and described differently, and as would be understood by a person of ordinary skill in the art. It is understood that none of the embodiments disclosed herein are limited to these nouns, and these element numbers can further include other words that would be understood by a person of ordinary skill reading and reviewing this disclosure in its entirety.

| | |
|---|---|
| X10 | flexible member, flexible connector, |
| | tether, sutures, cables, springs, tape, |
| | and any of woven, braided, or flat, as |
| | examples |
| X15a | pushrod tool rod |
| b | pushrod tool handle |
| X20 | Anchor; anchoring assembly |
| X21a | centerline |
| b | cross section |
| c | Thread overlap |
| X22 | cavity |
| b | bore; internal pocket |
| c | threads, straight |
| X23 | Staple; means for anchoring; anchoring |
| | member |
| a | projection |
| b | Inner shoulder |
| c | base |
| X24a | Chute or groove; passageway for |
| | tether and pad |
| b | entrance; exit |
| X26 | Body; fastener; means for anchoring; |
| | anchoring member |
| b | outer surface |
| c | tapered threads |
| d | tip |
| e | Outer diameter |
| f | Thread minor diameter |
| g | Wall thickness |
| h | Conical taper angle |
| X30 | head |
| b | undersurface |
| X32 | tether ring groove |
| X33a | angled expansion face |
| b | locking ring groove |
| X34a | recess for buckle, fastening feature, |
| | receptacle, or locating feature |
| b | buckle shape |
| X35 | flattened surface; sidewall |
| X36 | pin |
| b | hinge member |
| X38a | groove pins |
| b | locking interference pin |
| c | anti-rotation interference pin |
| X39 | slots |
| X40 | set screw |
| X42 | top surface |
| X44 | bottom surface |
| X46 | driver shape |
| X48 | tool guiding bore |
| X50 | pad |
| X52a | upper screw-contacting surface |
| b | pocket edge |
| X54a | lower tether contacting surface |
| b | concave surface |
| X56a | lateral groove-engaging surface |
| b | upward walls |
| X58 | compression pad |
| a | upper screw contacting surface |
| b | lower tether contacting surface |
| c | aperture |
| d | ears |
| X60 | buckle; securement member; |
| | connector for end of flexible member |
| a | outer shape |
| X62 | tether docking, mooring location; |
| | attachment location for flexible member |
| X70 | tether ring |
| X72 | body |
| X73a | angled compression face |
| b | locking ring groove |
| X74 | tether docking, mooring location; |
| | attachment location for flexible member |
| X75 | sidewalls; flattened surface |
| X76 | slot for arm |
| X78 | ledge for receiving arm lip |
| X82a | spring arm |
| b | circumferential split |
| c | fixed end |
| d | free end |
| X84 | ring tabs |
| b | locking tab |
| b2 | ramping face |
| b3 | abutting face |
| c | alignment tab |
| X90 | arm |
| X92 | hinge member |
| X93 | lengthwise arm section |
| X94 | lip for coupling to ledge |
| X95 | locking screw |
| X96 | driver shape |
| X97 | locking ring |

### DETAILED DESCRIPTION OF ONE OR MORE EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates. At least one embodiment of the present invention will be described and shown, and this application may show and/or describe other embodiments of the present invention, and further permits the reasonable and logical inference of still other embodiments as would be understood by persons of ordinary skill in the art.

It is understood that any reference to “the invention” is a reference to an embodiment of a family of inventions, with no single embodiment including an apparatus, process, or composition that should be included in all embodiments, unless otherwise stated. Further, although there may be discussion with regards to “advantages” provided by some embodiments of the present invention, it is understood that yet other embodiments may not include those same advantages, or may include yet different advantages. Any advantages described herein are not to be construed as limiting to any of the claims. The usage of words indicating preference, such as “various embodiments” or “preferably,” refers to features and aspects that are present in at least one embodiment, but which are optional for some embodiments, it therefore being understood that use of the word “preferably” implies the term “optional.”

The use of an N-series prefix for an element number (NXXXX) refers to an element that is the same as the non-prefixed element (XX.XX) except as shown and described. As an example, an element 1020.1 would be the same as element 20.1, except for those different features of element 1020.1 shown and described. Further, common elements and common features of related elements may be drawn in the same manner in different figures, and/or use the same symbology in different figures. As such, it is not necessary to describe the features of 1020.1 and 20.1 that are the same, since these common features are apparent to a person of ordinary skill in the related field of technology. Further, it is understood that some features 1020.1 and 20.1 may be backward compatible, such that a feature of a later discussed embodiment (NXX.XX) may include features compatible with other various embodiments that were discussed earlier (MXX.XX), as would be understood by those of ordinary skill in the art. This description convention also applies to the use of prime (′), double prime (″), triple prime (′″) and star or asterisk (*) suffixed element numbers. Therefore, it is not necessary to describe the features of 20.1, 20.1′, 20.1″, 20.1′″ and 20* that are the same, since these common features are apparent to persons of ordinary skill in the related field of technology.

Although various specific quantities (spatial dimensions, temperatures, pressures, times, force, resistance, current, voltage, concentrations, wavelengths, frequencies, heat transfer coefficients, dimensionless parameters, etc.) may be stated herein, such specific quantities are presented as examples only, and further, unless otherwise explicitly noted, are approximate values, and should be considered as if the word “about” prefaced each quantity. Further, with discussion pertaining to a specific composition of matter, that description is by example only, and does not limit the applicability of other species of that composition, nor does it limit the applicability of other compositions unrelated to the cited composition.

What follows are paragraphs that express particular embodiments of the present invention. In those paragraphs that follow, some element numbers are prefixed with an “X” indicating that the words pertain to any of the similar features shown in the drawings or described in the text. However, those of ordinary skill in the art will recognize various other non-X prefixed element numbers that discuss features applicable to other embodiments.

This document may use different words to describe the same element number, or to refer to an element number in a specific family of features (NXX.XX) It is understood that such multiple, different words are not intended to provide a redefinition of any language herein. It is understood that such words demonstrate that the particular feature can be considered in various linguistical ways, such ways not necessarily being additive or exclusive.

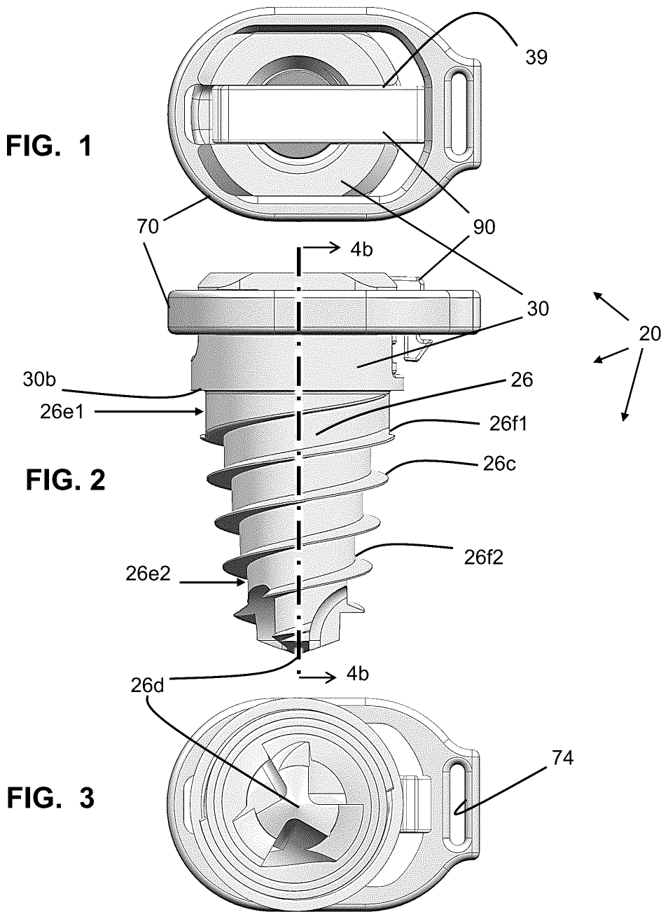

FIGS. 1-9 depict an apparatus 20 adapted and configured for fixation to a bone. In some embodiments, the apparatus is especially configured for fixation to cortical bone of a vertebrae. Still further embodiments include various features and components that permit multiple anchors 20 to be attached to different vertebrae with a flexible connection being established that is placed in tension between different anchors. In different embodiments, the flexible connections are established by flexible members such as sutures, tethers, springs or wire, as non-limiting examples. The anchors and the flexible connections are adapted and configured for establishing tension forces between the anchors, and preferably not supporting compression in the flexible member.

FIGS. 1, 2, and 3 show various orthogonal, external views of an anchoring apparatus 20 according to one embodiment of the present invention. Anchor 20 includes a head 30 and a body 26, with a tethering ring 70 coupled to the head. Body 26 extends from just underneath head 30 to a tip 26d. In some embodiments body 26 preferably includes a shaft having an outer surface 26b with tapered threads 26c. Preferably, threads 26c are adapted and configured for fixation to cortical-type bone material, although in yet other embodiments the threaded outer surface can include non-tapered threads configured for fixation to cortical and/or cancellous-type bone material, or any type of bone attachment scheme including non-threaded attachments. In some embodiments, the overall shape and size of anchor 20 is configured to have a small profile when fixed to vertebrae.

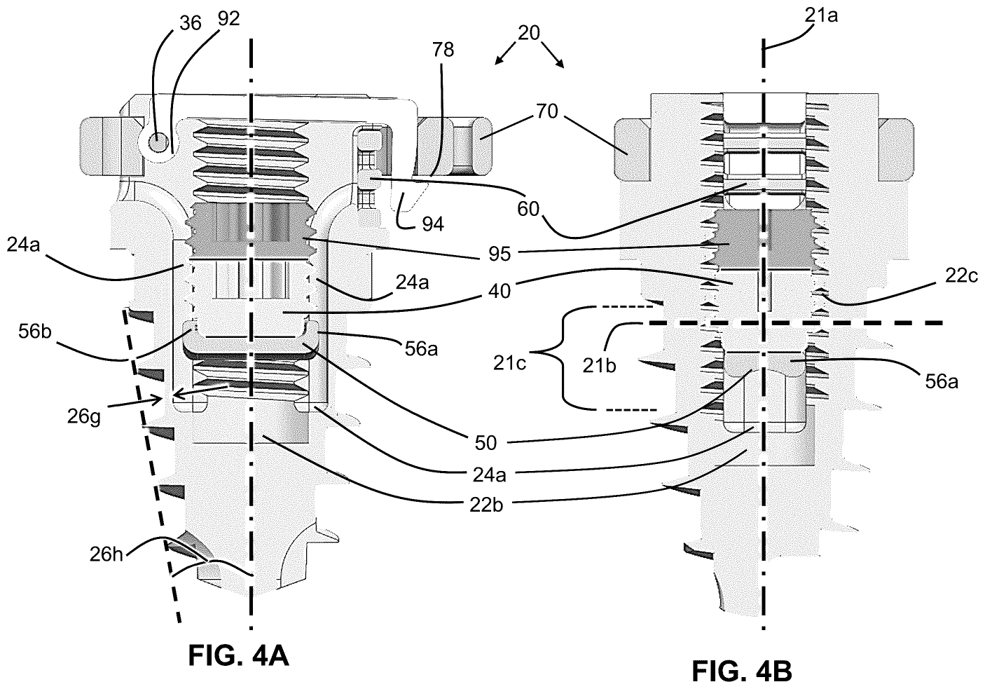

Referring to FIGS. 4A and 4B, various features internal to anchor 20 can be seen. Preferably, body 26 of anchor 20 includes an internal cavity 22 extending from the top surface of head 30 to an internal location proximate to tip 26d. This internal cavity preferably includes a generally cylindrical bore including a threaded section extending from the top of head 30 to an inner location in the bore 22b proximate to tip 26d. As illustrated in FIG. 4B, both the external tapered threads 26c and the internal straight threads 22c are contiguous and overlap within a length 21c of the body. Cross Section 21b identifies a part of this region. At this cross sectional location, anchor 20 has both external threads and internal threads, these threads each preferably being coaxial about a centerline 21a.

In some embodiments, the internally threaded section is interrupted on opposite sides by chutes or passageways 24a that extend from respective entrances 24b located in the head section, and extending axially downward toward the bottom of bore 22b (as best seen in FIG. 7). These passageways and entrances are adapted and configured for sliding contact with a flexible member, such as a tether, suture, or other. In some embodiments, these passageways extend through the internal threads 22c, such that the resulting internal threads are provided into separated, opposing sets of threads. Preferably, these passageways extend completely through the threads 22c, such that the distal end of the chutes are open to the distal end of the bore 22b. Preferably, the chutes 24a extend to the top surface of head 30 as corresponding slots or grooves 39.

Referring to FIG. 2, it can be seen that the body 26 of anchoring assembly 20 has a generally tapered shape to it in some embodiments. Body 26 has a first outer diameter 26e1 that reduces in size to a smaller outer diameter 26e2 proximate to tip 26d. Likewise, on those embodiments that have external threads, the minor diameter 26f1 toward the head 30 is larger than the minor diameter 26f2 that is proximate to tip 26d.

Referring to FIG. 4A, a tapering shape according to one embodiment of the present invention is illustrated by the conical taper angle 26h between the centerline of the body and a dotted line superimposed on the major diameter 26f of the threads. In some embodiments, both the major diameter of the threads, the minor diameter of the threads, and the outer diameter 26e all reduce in a distal direction from the head toward the tip 26d. This tapering extends generally from the underside 30b of the head. It has been found that this tapered shape is useful in packaging of the overall anchor assembly so as to achieve the tensioning increase and slack reductions discussed herein, while maintaining a low overall implanted height above the bone surface, and further achieving a sufficient grip onto the bone.

Another aspect of this tapered shape pertains to the internal pocket 22b and the grooves 24a. Referring to FIG. 4A, it can be seen that the internal pocket is substantially cylindrical, preferably including machine threads for reception therein of a set screw with machine threads. Preferably, the internal pocket has a length from the entrance or exit 24b to the bottom of the internal pocket that is of sufficient depth to retain within the pocket a sufficient length of flexible member to provide options to the surgeon during implantation. Further, it is helpful that the overall height of the anchoring system (from the tip to the top of the head) to be short enough to be useful in various different situations. For these reasons, it is helpful to have the internal pocket as deep within the body 26 as possible. However, the minimum wall thickness 26g needs to be configured to minimize any local stress concentrations (such as from bending stresses of the anchor relative imposed by the moment created between the bone attachment and the tension force of the flexible member), as well as to facilitate machining of the internal threads.

Although what is shown and described herein are bodies 26 that have a substantially uninterrupted taper (and corresponding reduction in minor diameter) substantially from the head to the tip, the present invention is not so constrained, and further includes those embodiments in which the body 26 has an overall shape that includes combinations of multiple conical shapes, and/or combinations of cylindrical and conical shapes. As one example, in some embodiments, the outer diameter of the body 26 is substantially cylindrical to a position proximate to the wall thickness 26g of FIG. 4A, and conically tapered thereafter to the tip.

Referring back to FIGS. 4A and 4B, the internal bore 22b is preferably populated with a set screw 40 and a locking screw 95, each in threaded engagement with threads 22c. Optionally, a pad 50 is located on the underside of the set screw 40. The set screw 40 and locking screw 95 preferably include straight threads that match the shape of threads 22c. Each screw 40 and 95 preferably includes a driver shape 46 or 96, respectively, on the top side of a respective screw, by which the surgeon can apply torque from a tool having a complementary driving shape.

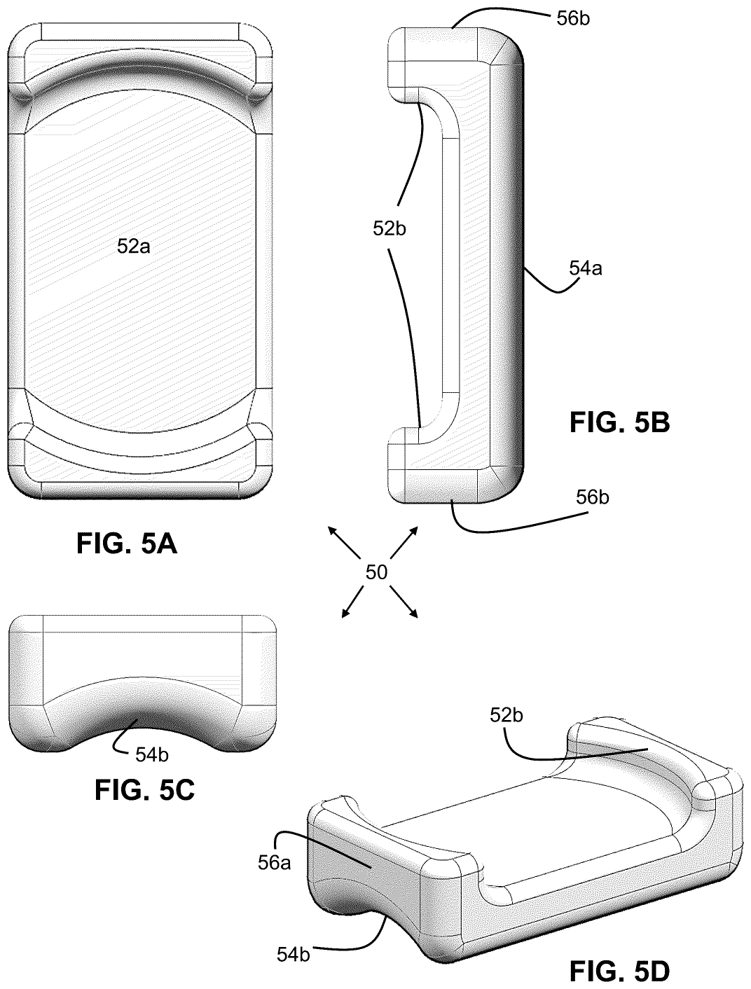

FIGS. 5A, 5B, 5C, and 5D depict various view of a pad 50 according to one embodiment of the present invention. Pad 50 in some embodiments preferably has a tray-type shape, including a central surface 52a configured for contact with the bottom surface 44 of set screw 40. Preferably, a pair of opposing and upstanding edges 52b provide lateral boundaries to the screw contacting surface 52a. Pad 50 further includes a lower surface 54a adapted and configured for sliding contact with the flexible member. In some embodiments, contacting surface 54a includes a concave section 54b that is preferably located centrally on the underside, and extending between the lateral surfaces 56a adapted and configured for sliding contact with a respective chute or passageway 24a. The upstanding walls 56b that engage the chutes 24a are best seen in FIGS. 5B and 5D.

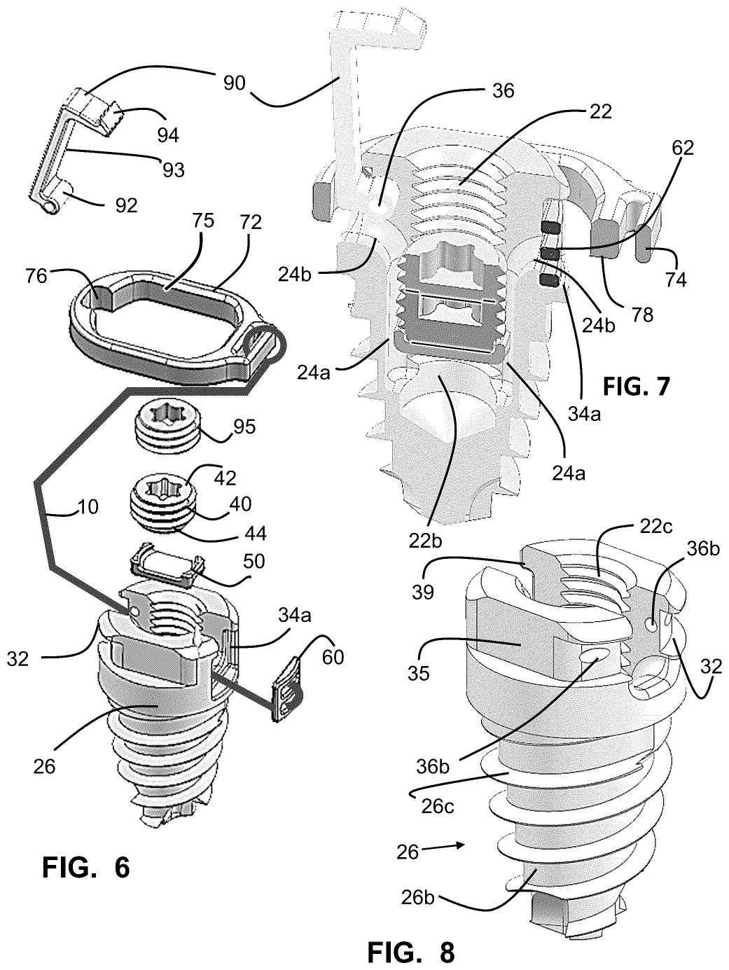

FIGS. 6, 7, and 8 show different aspects of head 30 and tethering ring 70. Head 30 preferably includes a groove 32 extending at least partially around the circumference of head 30 which is adapted and configured for placement therein of tethering ring 70. As best seen in FIGS. 6 and 8, the groove 32 preferably includes upper and lower walls that are spaced apart by a distance slightly greater than the height of ring 70. These upper and lower walls interfere with any attempt to remove ring 70 after it has been assembled onto head 30. Head 30 further includes one or more flattened surfaces 35 that are adapted and configured to receive there across complimentary-shaped surfaces of ring 70, and preferably to prevent rotation of ring 70 about head 30 once ring 70 is installed.

Referring to FIGS. 6 and 7, head 30 preferably includes a locating feature 34 that is adapted and configured to retain in position a separable member 60. A comparable securement member 160 can be seen in the side view of FIG. 11B. In one embodiment, member 60 has a type of buckle shape with a mooring location 62 for attachment to a flexible members 10. In a preferred embodiment, head 30 includes a recessed shape 34a that is complimentary to the external perimeter shape of buckle 60. However, various other embodiments of the present invention contemplate any manner of retaining or locating the position of tether securement member 60 onto the head of the anchor.

In some embodiments, the flexible member X10 is coupled to tether docking location X62, with the remainder of the tether going into the nearby entrance 24b, through the chutes 24a and out the opposing entrance 24b. Once the flexible member X10 is placed in tension, the securement member X60 is held against the outer surface 26b of the body, and retained in a particular location in the corresponding recess X34a.

FIGS. 6 and 7 depict exploded and non-exploded views, respectively, of a tethering ring assembly 70. Ring assembly 70 includes a body 72 that is adapted and configured to fit over the top of head 30. Body 72 includes an oblong internal section having a pair of substantially straight opposing sidewalls 75. One end of ring 70 includes a slot 76 adapted and configured to receive therein the hinge section of arm 90. Opposite to the slot 76 is a mooring location 74 adapted and configured for attachment to the flexible member 10. In one embodiment, the mooring location 74 includes a cross member that extends outwardly from a section of body 72 that preferably includes a ledge 78 for receiving in interlocking relationship the free end 94 of arm 90. Preferably the mooring locations 74 and 62 are adapted and configured to be attached to the end of a separate section of flexible member 10. As one example, the free end of a section of tether can be wrapped around with the end of tether sewn or otherwise attached onto another portion of the segment of tether.

The head 30 of anchor 20 further includes features for pivotal attachment of arm 90. As best seen in FIGS. 6 and 7, arm 90 includes a pivotal connection end 92 separated by a length of arm section 93 to a clasping end that includes a lip 94 for coupling to the underneath of tether ring ledge 78. The pivoting end 92 of arm 90 is pivotally coupled to head 30 by a pin 36 captured within head hinge receiving member 36b. FIG. 7 shows this pivoting, pin coupling of arm 90 to head 30. It can be seen in FIG. 7 that the slot 76 provides sufficient clearance for arm 90 to be pivoted upward and away from the top surface of head 30.

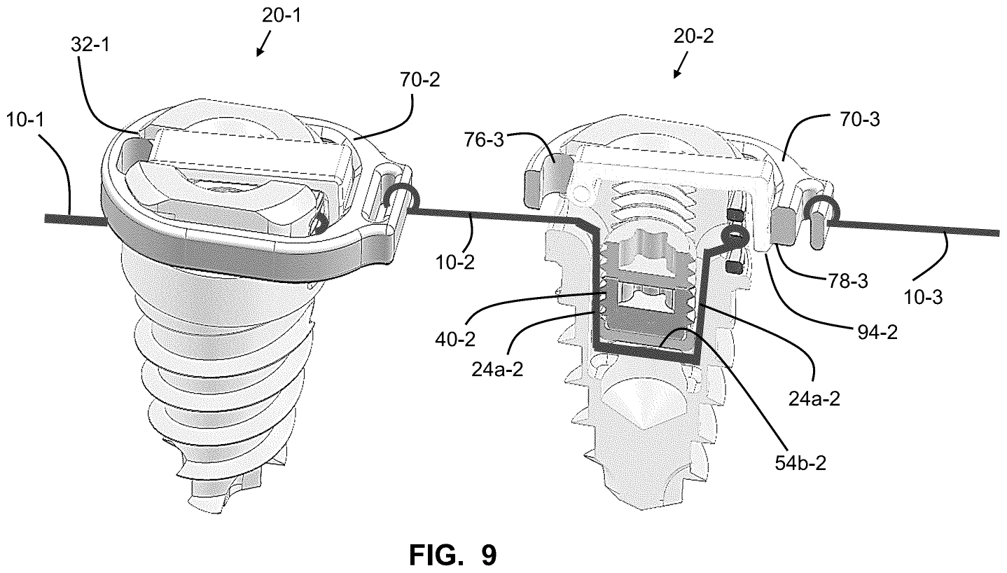

Referring to FIGS. 6-9, the use of pivoting arm 90 to releasably connect ring 70 to a head 30 will now be explained. A separated ring 70 is placed over the top of the head 30 of an anchor 20, with the pivoting arm 90 being rotated to an upwardly extending free state. The ring 70 is positioned such that cut out or slot 76 is located proximate to hinge member 92. The installed but unfastened ring initially sits on the bottom wall of groove 32 that is adjacent to the flattened surfaces 35 of head 30. Initially, there is a gap between the portions of groove 32 proximate to hinge member 36b, such that the ring body 72 is not yet captured between the upper and lower walls of groove 32. However, the internal, oblong shape of ring 72 is slightly greater than the corresponding distance of head 30, such that the unfastened ring body 72 can slide laterally (with flattened surfaces 35 each being in opposition to, and in sliding contact with, a corresponding flattened side wall 75), which allows for the initially installed ring 70 to be moved laterally until portions of ring 70 are captured within groove 32. This is best shown in FIG. 9, where the ring 70-2 is shown extending within groove 32-1.

With ring 70 being slid laterally for coupling within groove 32, the pivoting arm 90 is then pivoted downward until lip 94 at the free end of arm 90 is pushed downward such that it locks against the under ledge 78 of the ring. This is best seen in FIG. 9 with regards to the attachment of arm 90-2 to ring 70-3. In some embodiments, this placement of the free end of arm 90 into contact with the top side of ledge 78 will automatically slide body 72 in a direction such that hinge member 92 is closely captured within slot 76.

FIG. 6 presents an exploded view of an anchor assembly 20 according to one embodiment of the present invention. Body 26 receives in the central threaded bore first a pad 50, followed by a set screw 40, and then a locking screw 95. A section of a flexible member 10 such as a tether is connected at one end to a mooring location 62 on separable securement member 60, and at the other end to a mooring location 74 of ring 70. The section of tether extends from mooring location 62 (referring to FIG. 7) into an entrance 24b of body 26 that leads to a chute or passageway 24b. Referring briefly to FIG. 9 and anchor 20-2, the tether extends underneath pad 50 within concave undersurface 54b, into the opposing chute or passageway 24a, and out of the exit 24b opposite to the entrance.

Referring to anchor 20-2 of FIG. 9, it can be seen that the location of set screw 40-2 establishes the depth and the length of the internal passage for the tether 10-2. If set screw 40-2 is placed further within the threaded internal bore of anchor 20-2, then either: (1) more length of tether 10-2 will be located within anchor 20-2; or, a desired state of tension between mooring location 74-2 and mooring location 62 will be achieved. As will be explained later, in some embodiments the ring 70 attached to one end of the tether is on a different anchor than the securement member 60, which couples to the other end of the same section of tether. Referring to FIG. 7, it can be seen that the extension of bore 22b and threads 22c deep within the interior of body 26 permits a relatively long internal pathway for the tethering member. Further, the adjustment of the set screw by a specific distance will result in a change in the tethering pathway of twice (2X) that specific distance.

FIG. 9 shows an assembly of adjacent anchors 20-1 and 20-2 according to one embodiment of the present invention. Preferably, the ring 70-2 from anchor 20-2 is coupled to the head 30-1 of anchor 20-1. Therefore, the flexible member or tether 10-2 of anchor 20-2 establishes, after suitable adjustment of set screw 40-2, a desired degree of tension between the two adjacent anchors. It can further be seen that the tether 10-1 of anchor 20-1 can be used for coupling to another anchor (not shown), and likewise a ring 70-3 from an anchor 20-3 (not shown) is coupled to head 30-2, preferably with a state of tension created in tether 10-3.

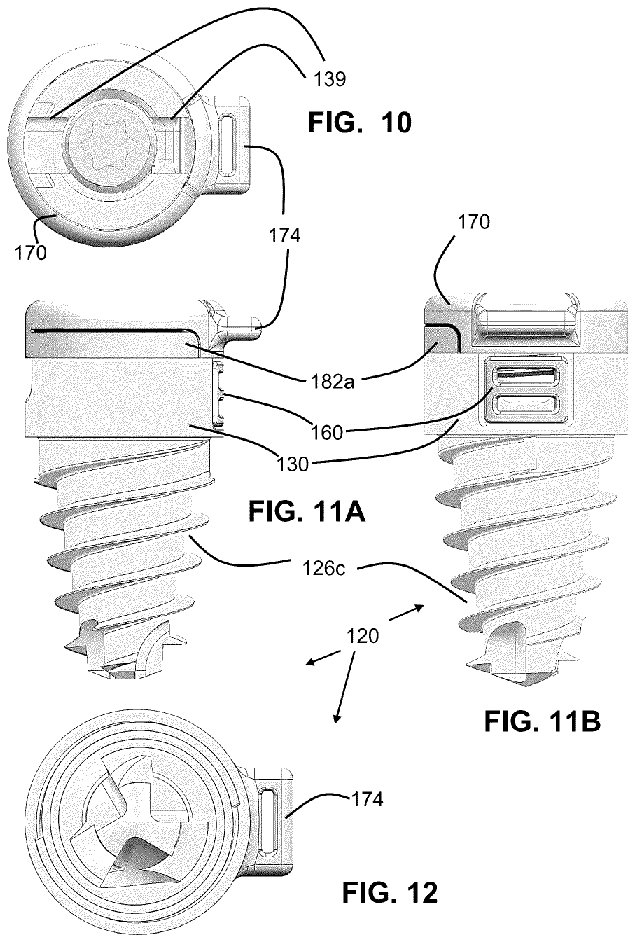

FIGS. 10-19 depict various views of an anchor 120 according to various embodiments of the present invention. As will be shown and described, anchor 120 includes a tethering ring 170 that interfaces with head 130 and operates differently with regards to interlocking in contrast to tethering ring 70 and head 30 previously described. However, as will be appreciated by persons of ordinary skill in the art, various other aspects and features of anchor 120 are the same or similar to various aspects and features of anchor 20.

As can be seen in FIGS. 10-12, anchor 120 includes a body 126 and a head 130. The outer surface 126b of body 126 includes tapered threads extending from a tip 126d to a location just underneath head 130. A securement member 160 adapted and configured for attachment to a flexible connector is retained on head 130 in substantially the same manner as described with regards to member 60 on head 30. Preferably, the chutes 124a extend to the top surface of head 130 as corresponding grooves 139.

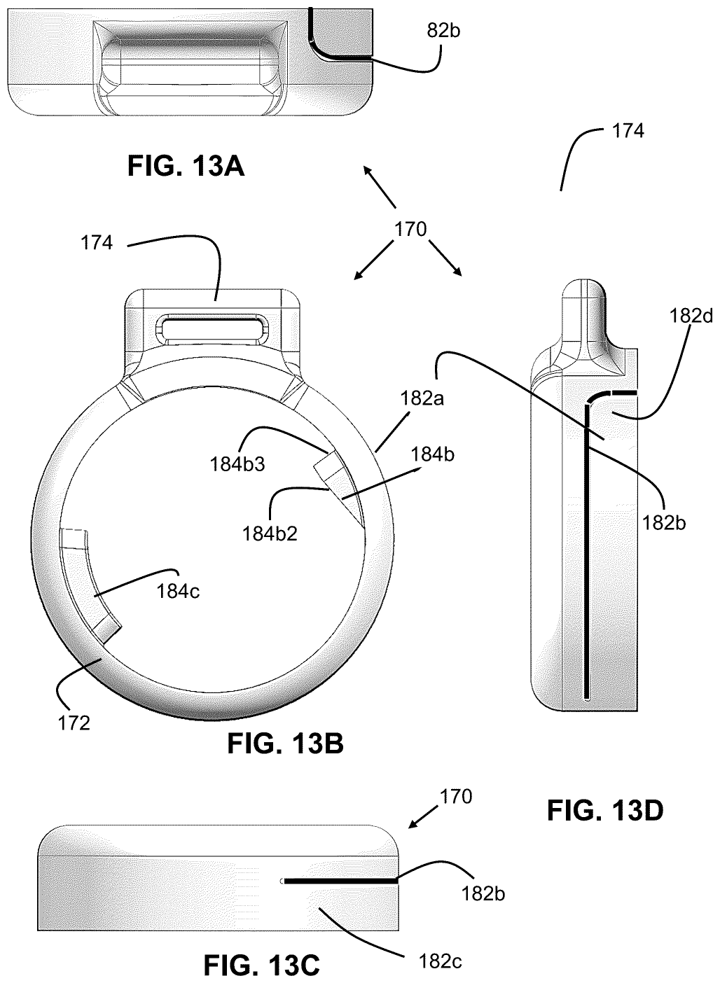

A tethering ring 170 according to another embodiment of the present invention is shown in FIGS. 13A, 13B, 13C, and 13D. One side of tethering ring 170 includes a mooring location 174 adapted and configured for attachment to an end of a flexible connector 110 (as best seen in FIG. 15). As previously discussed, all of the embodiments shown herein contemplate any manner of attachment of the end of a flexible connector to any of the mooring locations, including by sewing, heat fusion, ultrasonic fusion, or by use of fasteners (such as rivets), all as non-limiting examples. In some embodiments, the body 172 of ring 170 is a one-piece ring, and continuous about the circumference. However, yet other embodiments of tethering ring 170, or any of the rings X70 shown herein, can be fabricated in single pieces or multiple pieces, and in any manner, and from any type of biocompatible material.

In some embodiments, a section 182a of ring 170 is adapted and configured to function as a cantilevered spring. As best seen in FIGS. 13A, 13C, and 13D, a spring arm 182a is located on the groove-contacting underside of body 172. Spring arm 182a is defined by a split 182b in the material of body 172 that extends from a location proximate to mooring location 174 (as best seen in FIG. 13A), extending along one side of circumference of the ring body (as best seen in FIG. 13D), and terminating at a location generally opposite of mooring location 174 (as best seen in FIG. 13C). In some embodiments, this split is accomplished by a method such as wire EDM, although various embodiments contemplate separation of the integral cantilever arm in any manner, and still further contemplate the use of a spring arm that is separable from the ring, and attached with a hinge and/or spring.

Referring to FIG. 13D, it can be appreciated that spring arm 182a has a free end 182d capable of maximum displacement that is proximate to mooring location 174, and an attachment end 182c depicted toward the bottom of FIG. 13D that operates as a cantilever-type spring attachment. Although what has been shown and described is a spring arm having an angular extent greater than 90 degrees with a free end located near a tether mooring attachment, it is understood that various other embodiments of the present invention contemplate yet other types of spring arms, and these embodiments are not constrained by the spring arm shown and described. Still further, various other embodiments include a locking tab 184b that is spring loaded relative to the body 172, and capable of displacement into a pocket (not shown) of body 172.

Referring to FIG. 13B, it can be seen that the inner diameter of ring body 172 includes one or more tabs 184 adapted and configured for coupling of ring 170 to head 130. In the embodiment shown, the spring arm 182 includes a locking tab 184b that is adapted and configured to interlock with a locking interference pin 138b that will be described later. Locking tab 184b is coupled to spring arm 182a, and the movement of the end 182d and tab 184b is interdependent in a fixed relationship. It can be seen that tab 184b includes a shape that is somewhat triangular, including an angled, ramped side that extends from the inner diameter of spring arm 182a at an included angle that is less than about sixty degrees. This ramping face 184b2 terminates on one side with an abutting face 184b3 that is generally perpendicular to the inner diameter of spring arm 182a. As will be shown and described later, ramping face 184b2 interacts with a corresponding interference pin 138b to move the free end of arm 182a in a radially outward direction. Abutting face 184b3 interacts with that same pin to prevent rotation in one direction.

Ring body 172 further includes a second, alignment tab 184c that, in some embodiments, is located generally opposite of locking tab 184b. Preferably, the angular relationship of tabs 184b and 184c is the same as the angular relationship of slots 139 in head 130. Alignment tab 184c extends from the inner diameter of body 172 radially inward across an angular sector preferably less than about 45 degrees. Alignment tab 184c interacts with a pin 138c in a groove of head 130 to prevent axial separation of ring 170 from head 130, as will be described later.

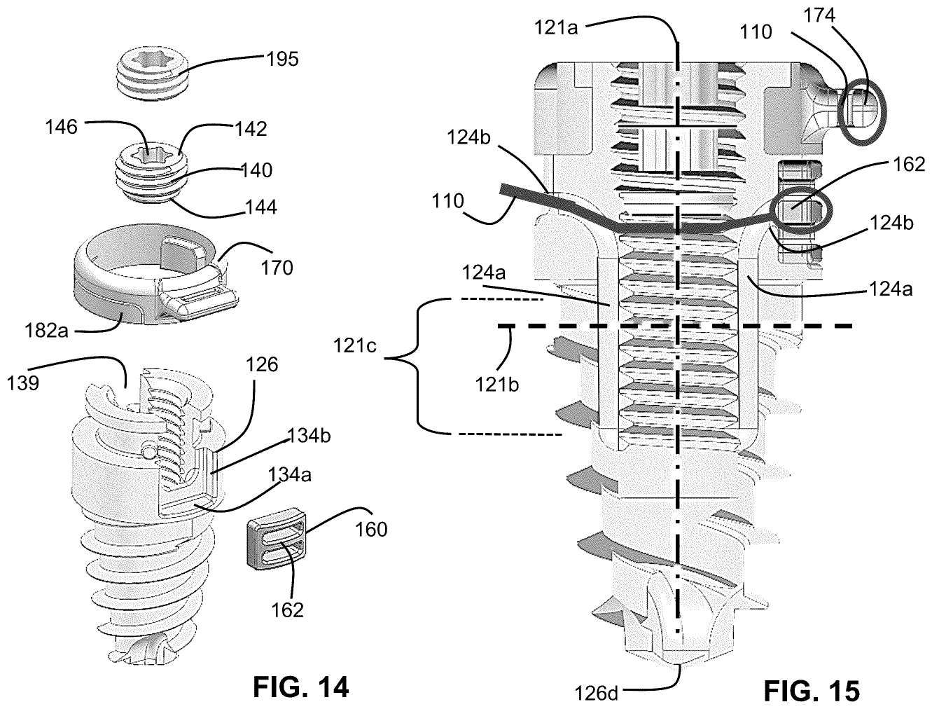

FIGS. 14 and 15 depict exploded and cross sectional views of anchoring assembly 120. It can be seen that body 126 includes an outer surface with tapered threads 126c that are coaxial about the centerline 121a with a section of threads 122c within an internal bore 122b. Body 126 includes a region 121c where the internal threads and external threads are contiguous, with the cross section at line 121b being an example of this region threaded on both the outside (tapered threads) and inside (straight threads).

Further, and also similar to anchor assembly 20, a set screw 140 and locking screw 195 are threadably coupled within bore 122b. The bottom surface 144 of set screw 140 can establish the bottom of the path of the tether 110 (as shown in FIG. 15) although in other embodiments a pad 150 (not shown) can also provide the contact interface with the tether. It is understood that a pad X50 is optional in all embodiments shown and described herein. It is also understood that a locking screw X95 is optional in all embodiments, although in such embodiments it is preferable that other means for locking of set screw X40 be provided.

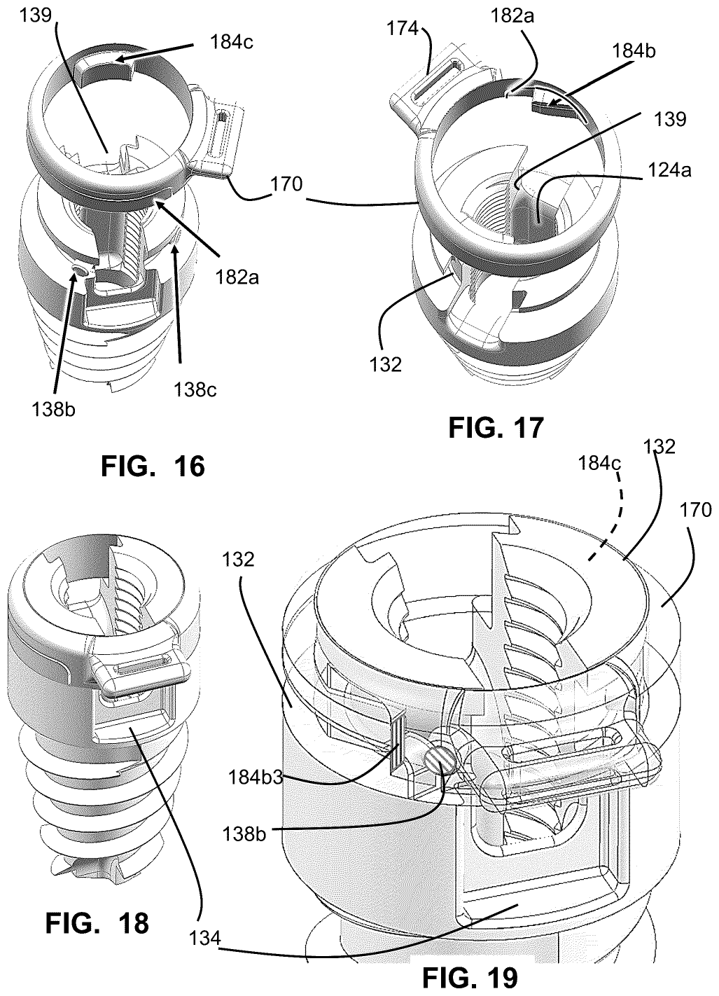

FIGS. 16-19 depict the interaction of ring 170 with head 130. FIGS. 16 and 17 show two views of the initial placement of ring 170 onto head 130. Ring 170 is initially placed upon head 130 by aligning each of the tabs 184b and 184c with a corresponding slot 139. FIG. 16 shows tab 184c placed directly above a slot 139, and FIG. 17 shows the opposite tab 184b being located above the opposite, each of the tabs 184 being sized to have a width that is less than the width of the corresponding slot 139 through which it fits. Still further, each of the tabs 184 have an angular, spatial relationship that is substantially the same as the angular and spatial relationship of the slots 139. In the embodiment of anchoring assembly 120, this angular relationship shows each tab about 180 degrees displaced from the other tab, and each slot likewise displaced. However, other embodiments of the present invention contemplate angular relationships other than 180 degrees. It is further noted that the slots 139 are geometric extensions of the chutes 124a, although it is noted that this relationship of having the slots and tether passageways being extensions of one another is not necessary, and yet other embodiments of the present invention contemplate having the tether slots displaced from the tether chutes angularly displaced from the slots at the top of the head.

The placement of ring 170 onto the bottom groove surface 132 establishes the axial location of ring 170 and head 130. After this initial placement, with the tabs initially placed in the corresponding slots, the ring is rotated relative to the head. Comparing FIGS. 16 and 19, it can be seen that the ring is rotated clockwise relative to the head. This rotation moves the ramping surface 184b2 of tab 184b over the top of pin 138b. This relative rotation results in an outward pushing of the outer surface of tab 138b against tab 184b, thus pushing ring arm 182a outward. As this rotation is continued, tab 184b is moved past pin 138b, as seen in FIG. 19. Any subsequent attempt to rotate ring 170 in a counterclockwise direction results in abutment of the side of pin 138b against the abutting face 184b3 of tab 184b. This abutment prevents counterclockwise rotation.

Referring to FIG. 19, tab 184c (not shown) is located generally opposite of tab 184b. With regards to alignment tab 184c, this tab is initially located in the corresponding groove 139. As ring 170 is rotated in a clockwise direction, tab 184c sits in a position between the top and bottom walls of groove 132. Likewise, the height of tab 184b is located between the top and bottom walls of groove 132. The top wall of groove 132 and the top surfaces of the tabs 184 thus interfere and prevent any axial removal of ring 170 from head 130.

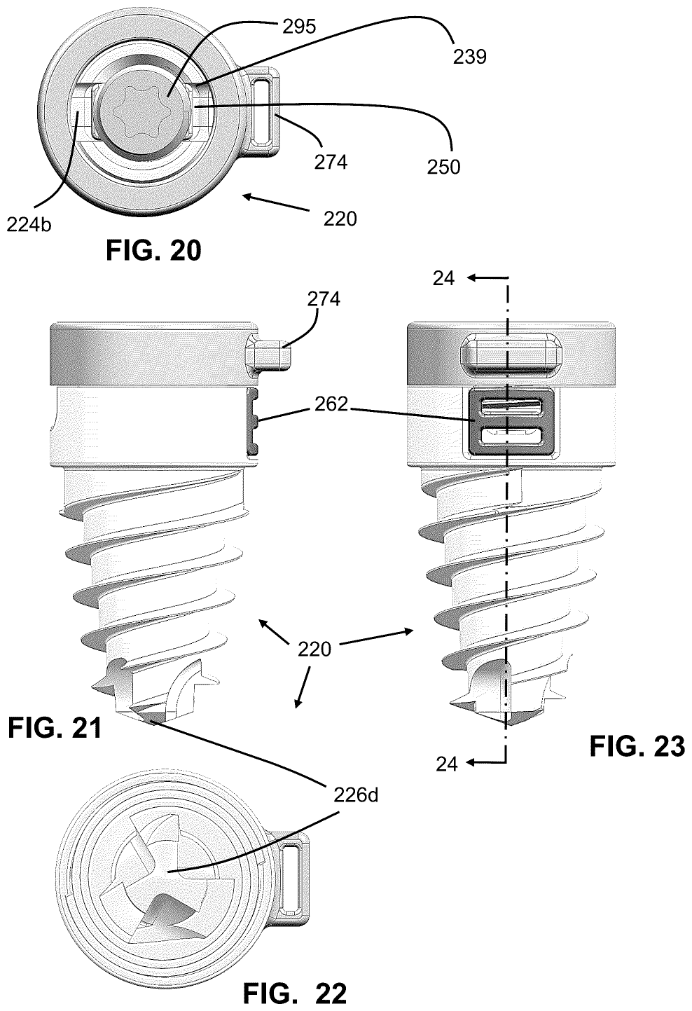

FIGS. 20-28 shows an anchoring assembly 220 that shares various components and features with the aforementioned anchoring assemblies 20 and 120. However, the ring 270 couples to head 230 in yet a different manner. Referring to FIGS. 20-23, it can be seen that anchoring assembly 220 includes a body 226 that includes a head 230 and a shaft having an outer surface 226b that preferably includes tapered threads extending from the head to a tip 226d. A securement member 260 having a mooring location 262 is retained on head 230 in a manner as previously discussed.

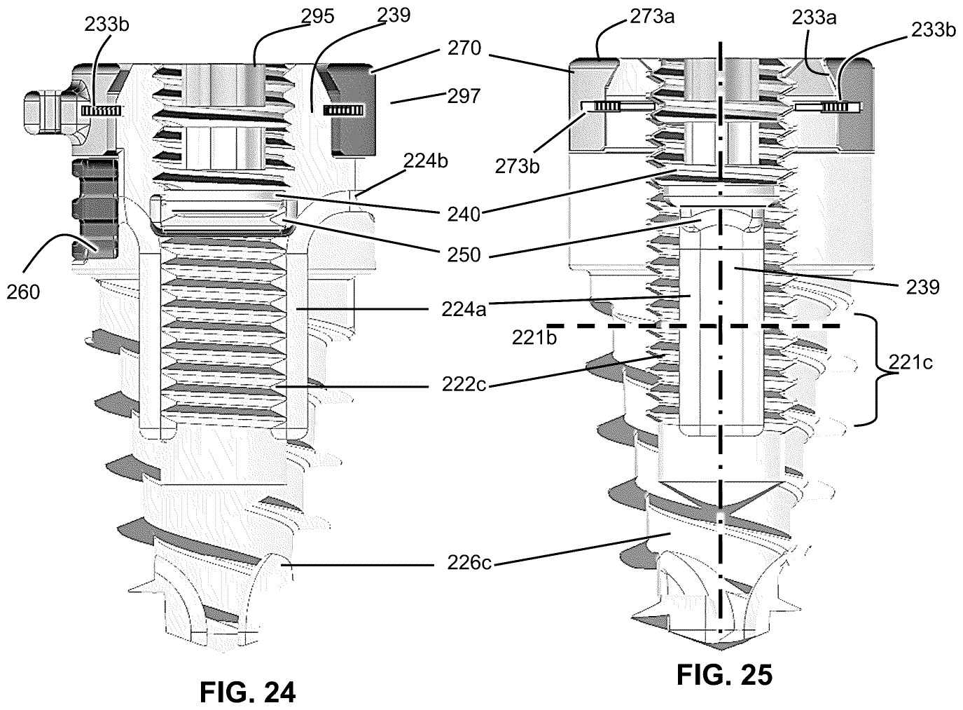

FIGS. 24 and 25 depict orthogonal cross sectional views of anchoring assembly 220. It can be seen that body 226 includes an outer surface with tapered threads 226 that are coaxial about the centerline 221a with a section of threads 222c within an internal bore 222b. Body 226 includes a region 221c where the internal threads and external threads are contiguous, with the cross section at line 221b being an example of this region.

Further, and also similar to anchor assemblies 20 and 120, a set screw 240 and locking screw 295 are threadably coupled within bore 222b. The bottom surface 244 of set screw 240 can establish the bottom of the path of the tether 210 (as shown in FIG. 15) although in other embodiments a pad 250 (not shown) can also provide the contact interface with the tether. It is understood that a pad X50 is optional in all embodiments shown and described herein. It is also understood that a locking screw X95 is optional in all embodiments, although in such embodiments it is preferable that other means for locking of set screw X40 be provided.

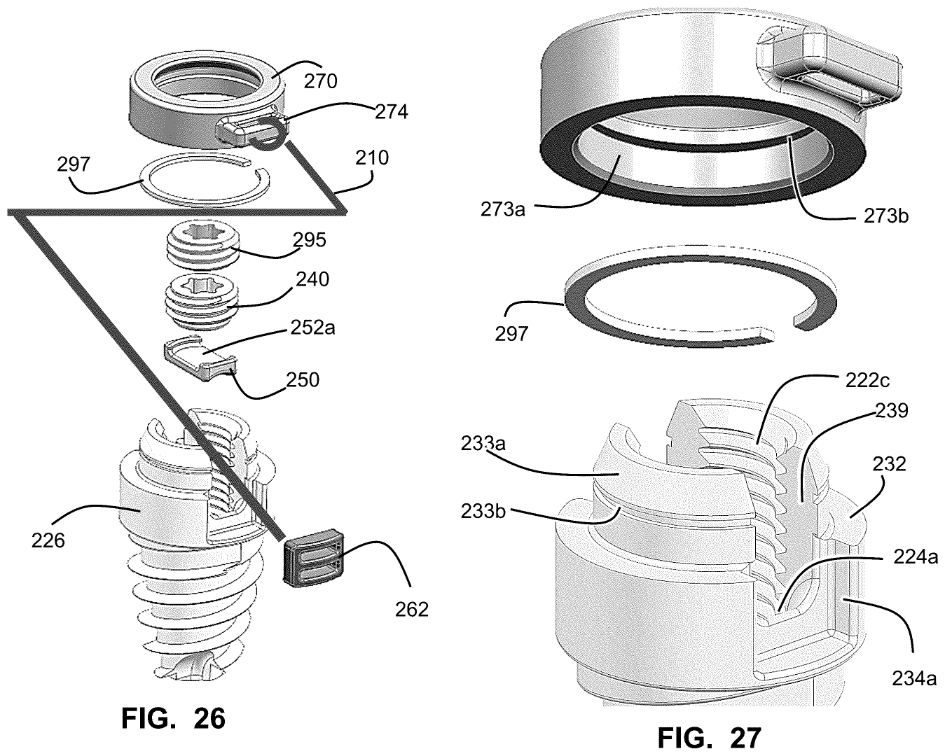

FIGS. 26 and 27 depict exploded views of anchor 220 that illustrate the coupling of ring 270 to head 230. It can be seen that ring 270 is preferably a unitary ring having a mooring location 274 for attachment to a tether 210. Preferably, the inner diameter of ring 270 includes a groove 273b that extends substantially around the entire circumference of the inner diameter. Head 230 includes a groove 233b that extends substantially around the inner outer diameter of the groove portion 232 of head 230. It is noted that groove 232 in some embodiments has only a lower wall, on which the lower surface of ring 270 resides after assembly.

Anchoring assembly 220 further includes a separable spilt locking ring 297. In some embodiments, ring 230 is a substantially circular ring that incorporates a gap between free ends of the ring. However, yet other embodiments of the present invention include split rings in which the free ends overlap. In those embodiments including a gap, the gap is preferably sized such that when ring 270 is fully compressed and the free ends touch, that the outer diameter of the compressed ring 297 is less than the inner diameter of the groove 273b of ring 270. Further, the inner diameter of the compressed ring 297 is slightly larger than the inner diameter of groove 233b. With this or a similar set of selected geometries, the ring 297 in its free state can reside within a placement of ring 297 concurrently within both groove 223b and groove 273b can be seen in the cross sectional views of FIG. 24 and FIG. 25. In this manner, locking ring 297 can reside in a free state or partially compressed state within both the inner groove 233b and the outer groove 273b, thus preventing axial separation of ring 270 from head 230.

In some embodiments, this assembly of head 230 and ring 270 is facilitated by conically-angled surfaces that assist in the compression of ring 297. Referring to FIG. 27, it can be seen that the underside of ring 270 includes a conically angled face 273a that is adapted and configured for sliding contact with the outer diameter of ring 297. Further, head 230 includes a conically shaped pair of expansion faces 233a that, in some embodiments, are split apart by the slots 239. This angled expansion face 233a is adapted and configured to have a topmost, smallest diameter end that is less than the inner diameter of ring 297 in its free state.

Preferably, but optionally, an anchoring assembly 270 is packaged and sold with the split ring 297 not being attached to either a head 230 or to a ring 270. After the user initially separates the unretained 270 from the accompanying body 230, the split ring 297 can then be inserted into either the groove 273b of the ring, or into the groove 233b of the head 230. After the body 226 of anchor 220 is secured to the bone, a loose tethering ring from one installed anchor can then be placed over and pushed downward onto the adjacent implanted anchor. As the tethering ring 270 and head 230 are compressed together, the angled surface 233a (for those cases in which the split ring is preassembled into the ring), or the angled mating surface 273a (for those embodiments in which the split ring is preloaded into the head) facilitate the expansion or contraction, respectively of the split ring as it couples to the corresponding groove.

In yet other embodiments pertaining to the assembly of ring 270 onto head 230, the uncompressed ring 297 is placed onto the conical surfaces 233a. The tethering ring 270 is then placed on top of head 230, and pushed downward. With this axial load, the angled face 273a pushes ring 297 downward onto surface 233a and toward groove 233b. As the ring moves over the expansion faces 233a, the ring expands until it is large enough to fit within groove 233b. Continued downward movement of ring 270 results in compression of the installed ring 297 until the outer diameter of ring 297 can fit within groove 273b. Once ring 297 is fit within both grooves 273b and 233b (regardless of the groove in which initial placement is achieved), the ring 297 will expand toward its free state. This installed locking ring 297 is thus captured in both grooves 223b and 273b, the captured ring 297 having an inner diameter too small to be removed from groove 233b, and an outer diameter too large to be removed from groove 273b.

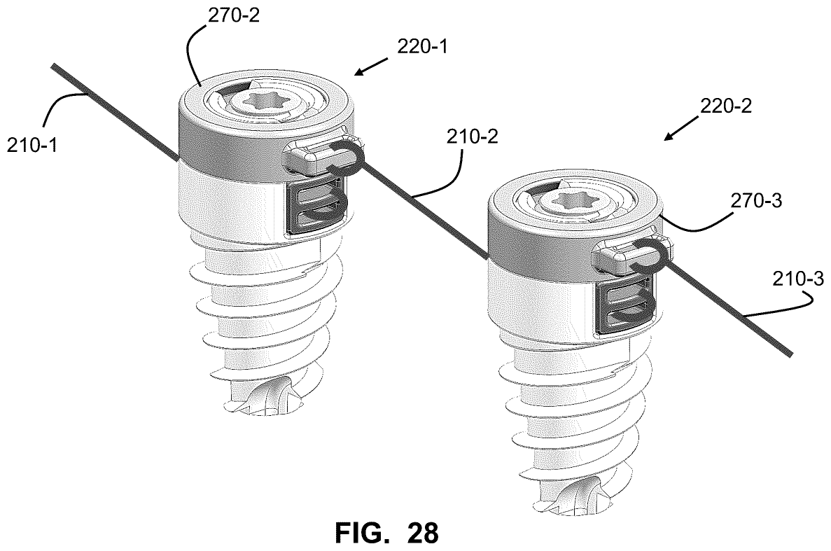

FIG. 28 shows an assembly of adjacent anchors 220-1 and 220-2 according to one embodiment of the present invention. Preferably, the ring 270-2 from anchor 220-2 is coupled to the head 230-1 of anchor 220-1. Therefore, the flexible member or tether 210-2 of anchor 220-2 establishes, after suitable adjustment of set screw 240-2, a desired degree of tension between the two adjacent anchors. It can further be seen that the tether 210-1 of anchor 20-1 can be used for coupling to another anchor (not shown), and likewise a ring 270-3 from an anchor 220-3 (not shown) is coupled to head 230-2, preferably with a state of tension created in tether 210-3.

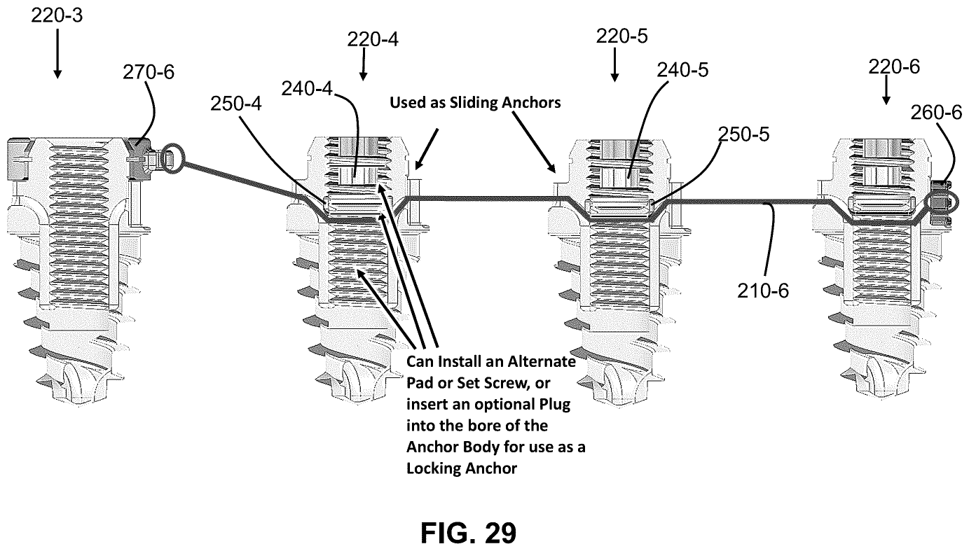

FIG. 29 shows an assembly of adjacent anchors 220-3, 220-4, 220-5, 220-6 according to another embodiment of the present invention. It can be seen that the tethering ring 270-6 from anchor 220-6 is coupled to the head of a different anchor 220-3. The flexible member 210-6 is sufficiently long enough to pass through one or more anchoring assemblies 220-4 or 220-5. Any of the set screws 250-4, 250-5, or 250-6 can be used to adjust the tension within flexible member 210-6.

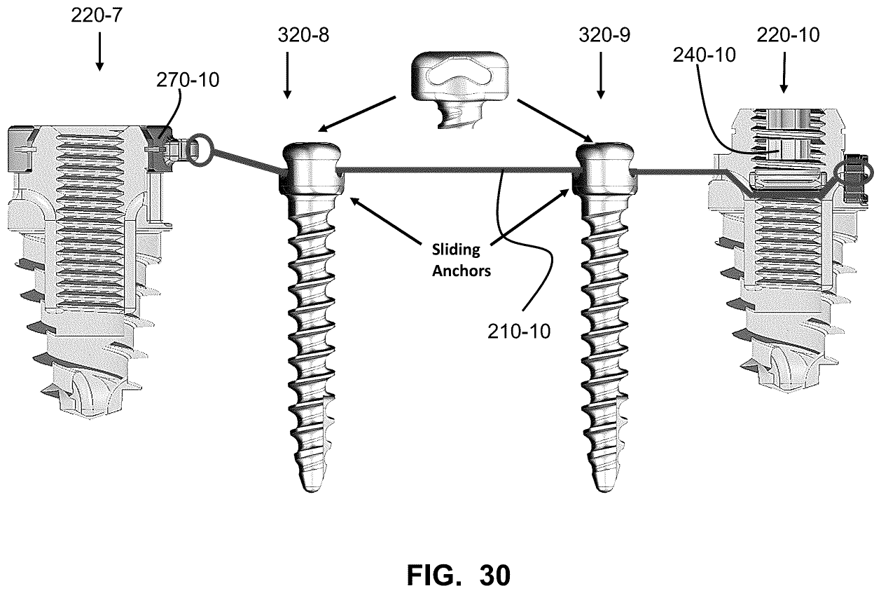

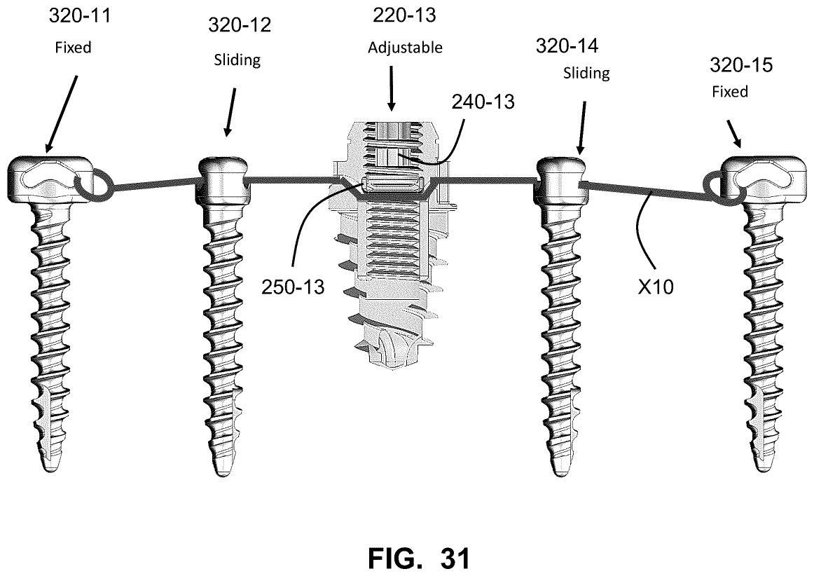

Referring to FIG. 30, there is shown an arrangement of adjacent anchors according to another embodiment of the present invention. FIG. 30 includes, as examples, anchoring assemblies 220-7 and 220-10 generally as discussed previously herein. Each of these anchors are placed on opposite ends of a tensioning tethering member 210-10. The tension in tether 210-10 is established by the position of set screw 240-10. Placed inbetween anchors 220-7 and 220-10 are tethering anchors 320-8 and 320-9, such as those disclosed in provisional patent application Ser. No. 62/623,769, filed Jan. 30, 2018, titled VERTEBRAL BODY TETHERING WITH SUTURE LOOPS, the portions of which pertaining to these same anchors being incorporated herein by reference. Tethering anchors 320-8 and 320-9, each attached to positions along the patient's spine provide guidance to the path of the flexible tethering member 210-10. The anchors 320 preferably do not alter the tension in the tether. It is understood that the guiding structures between anchors 220-7 and 220-10 can be of any type, attached to bone in any manner.

FIG. 30 shows an arrangement of tethering anchors according to yet another embodiment of the present invention. A section X10 of flexible member is attached to tethering anchors 320-11 and 320-15. In some embodiments, one or more guiding tethering anchors 320-12 and 320-14 can also be used to guide the path of the tether X10. In still further embodiments, an adjustable anchor 220-13 is placed between anchors 320-11 and 320-15, with the tether X10 passing within the passageways 224b-13, and underneath a set screw 240-13 and/or a pad 250-13. The surgeon can adjust the tension in tethering member X10 by altering the position of the set screw, as discussed previously.

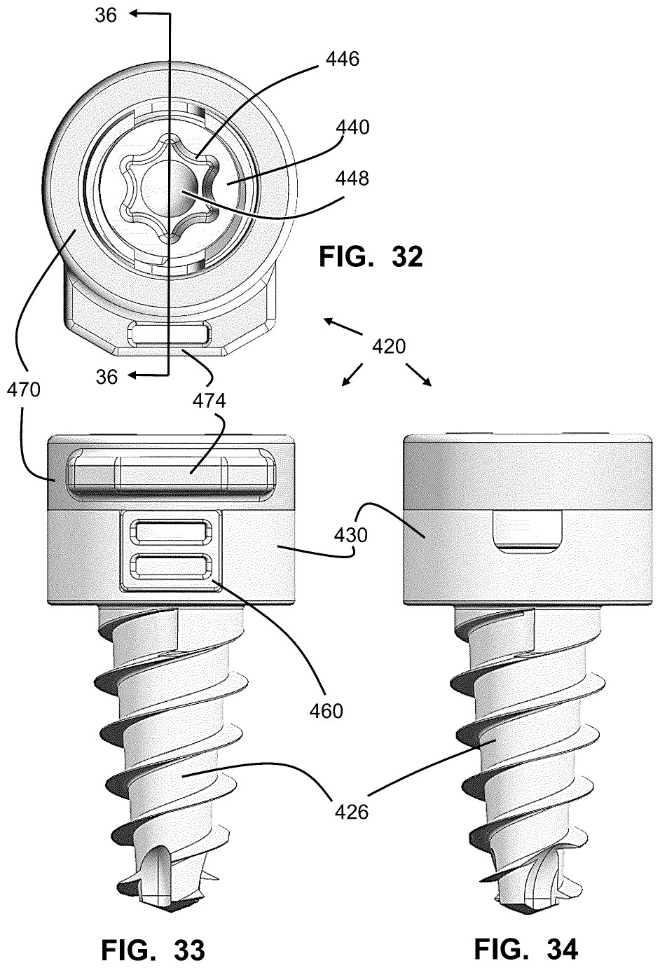

FIGS. 32-39 depict an anchoring assembly 420 that shares various components and features with the aforementioned anchoring assemblies 20, 120, and 220. In particular, the tethering ring 470 couples to head 430 in a manner similar to that for ring 270 on head 230. Further, the manner of securing a flexible tether to a buckle 460 and tethering 470 are generally as described before. However, the adjustment of tension in the flexible member 410 and the subsequent locking of the flexible member 410 in a position are accomplished differently.

Referring to FIG. 36, it can be seen that the internal pocket 422b within the interior of body 426 is preferably a smooth cylindrical bore, and preferably void of internal threads, although yet other embodiments contemplate non-smooth, non-cylindrical bores, and further contemplate internal pockets 422b being threaded for a coupling to set screw 440. Body 426 preferably includes a pair of entrances/exits 424b through which a flexible member 410 (not shown) can slide. These entrances/exits 424b lead into the internal pocket 422b.

Set screw 440 preferably includes a through bore 448 that preferably extends through the driver shape 446, and is in line of sight with internal pocket 422b.

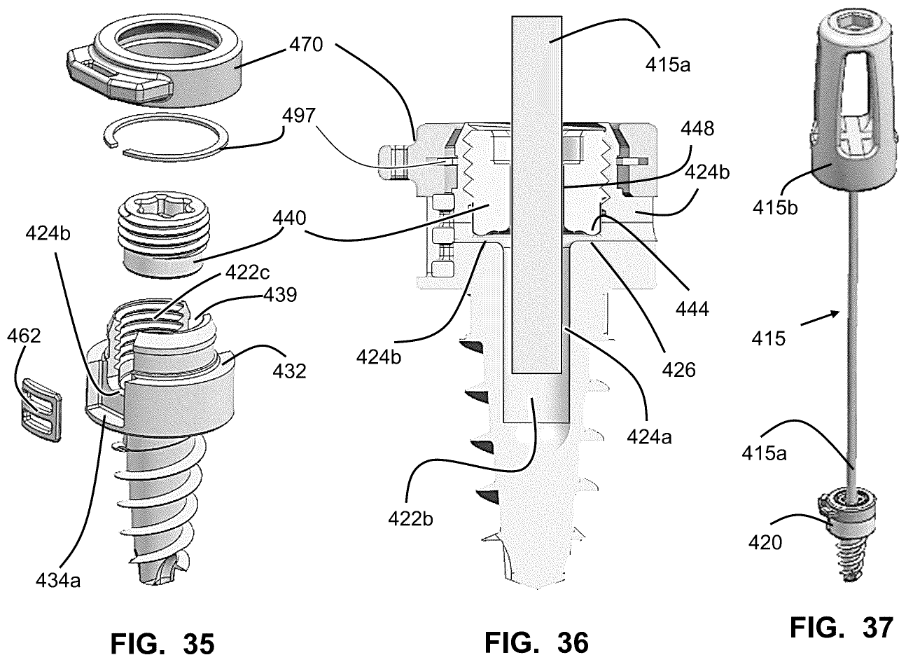

Preferably, set screw 440 can be threadably engaged with threads 422c as best seen in FIG. 35. Preferably, these threads extend for a sufficient distance within head 430 that set screw 440 can bottom out within head 430. Referring to FIG. 36, it can be seen that set screw 440 has a peripheral bottom surface 444 that preferably can contact an upwardly facing portion of body 426 proximate to entrances/exits 424b. Therefore, any flexible member 410 passing between body 426 can be contacted and compressed between these two opposing faces. With the configuration shown in FIG. 36, the user is able to fully compress or pinch the flexible member between the set screw and the body, although various other embodiments contemplate one or more abutments for preventing complete contact between the set screw and the body so as to avoid damage to the flexible member.

In an alternative embodiment, a separable compression pad 458 as shown in FIG. 39 is placed under set screw 440. Referring to FIG. 35, in some embodiments a compression pad 458 is placed between se screw 440 and entrance/exit 424b, located at the bottom of slots 439. Compression pad 458 preferably has a pair of opposing extended ears 458d that sit within the corresponding slots 439 and prevent rotation of compression pad 458. Pad 458 includes a central aperture 458c which is in general alignment with tool guiding bore 448 and internal pocket 422b. Referring to FIG. 36, in this alternative embodiment the compression pad 458 would be seen located between bottom surface 444 and the uppermost ledge of body 426, proximate to entrances 424b. In this alternative, when set screw 440 is tightened, the top surface 458a is in sliding contact with bottom 444, and the underneath 458b is in static contact with the tether as it comes in and out of the entrance/exit. Therefore, the tightening of set screw 440 does not grind or abrade against the tether, and the compression of the tether is between the non-rotating body 426 and compression pad 458.

Although FIG. 36 shows an arrangement in which the bottom face of the set screw and an upwardly facing ledge of the body can be tightened together so as to restrain sliding motion of the tether in or out of the entrance or exit, yet other embodiments contemplate other configurations for accomplishing a frictional restraint of the flexible connector. As one example, in some embodiments the bottom surface of the set screw is conically tapered, rounded, hemispherical, etc., such that advancement of the set screw can result in a compression and frictional restraint between the underside of the set screw and a corresponded rounded, conical, or other shaped edge of the internal pocket 422b adapted and configured to compress the flexible member but preferably not damage the flexible member. In yet other embodiments, the top facing surface of body 426 proximate to entrance/exit 424b can include a rounded groove or pocket that extends circumferentially within head 436, and having a shape complementary to that of the rounded corner edges of bottom surface 444. Such a mating of a rounded set screw edge within a rounded pocket can provide an alternative means to manage the state of stress within the compressed and restrained flexible connection.

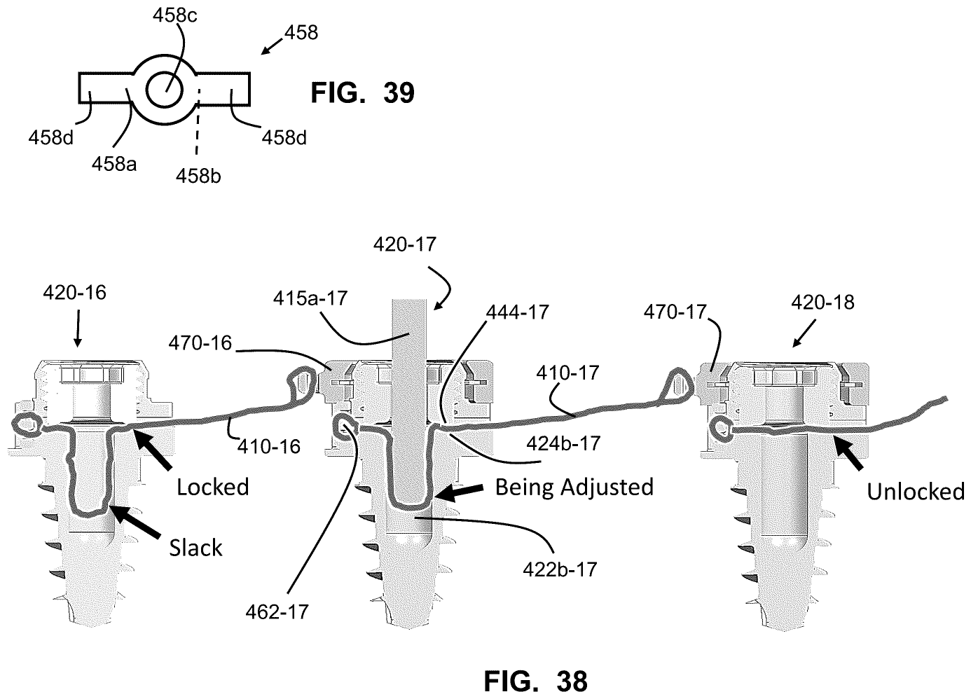

Adjustment of the tension in the flexible member and subsequent restraint of the tensioned flexible member will now be discussed with regards to FIGS. 36, 37, and 38. FIG. 38 shows a plurality of adjacent anchors 420-16, 420-17, and 420-18. It can be seen that the tethering ring and flexible member 470-16 and 410-16, respectively, extend from anchor 420-16 and coupled to adjacent anchor 420-17. Likewise, the tethering ring and flexible member 470-17 and 410-17, respectively, are shown coupled to adjacent anchor 420-18.

FIG. 38 shows the middle anchor 420-17 being adjusted. The flexible member 410-17 is attached at one end to mooring location 462-17, from which it enters an entrance 424b-17, passes downward into internal pocket 422b-17, extends upward out of internal pocket 422b-17, and then through the opposite exit 424b-17. This tether 410-17 then extends to a mooring location on tethering ring 470-17, which is now coupled to head 430-18.

The distal end 415a-17 of an adjustment tool 415 has been placed within the tool guiding bore 448-17 of set screw 440-17, and pushed into the interior pocket 442b-17. As the end of the tool extends through both the through bore and the internal pocket, the end of the tool pulls excess tethering material 410-17 into the bore, and wrapped around the distal end of the tool. In this manner, pushing of the tool end into the internal bore will create tension in flexible connector 410-17. Once an acceptable degree of tension is achieved, a driving sleeve (not shown) extending around the outer diameter of pushrod end 415a-17 is coupled into the driver shape 446-17. As the surgeon maintains an axial load on the pushrod to achieve the desired tension, the handle 415b (as best seen in FIG. 37) can be rotated to tightened set screw 440 in place, compressing and frictionally restraining the tether between the rounded undersurface 444 as the top facing ledge adjacent to the entrances 424b.

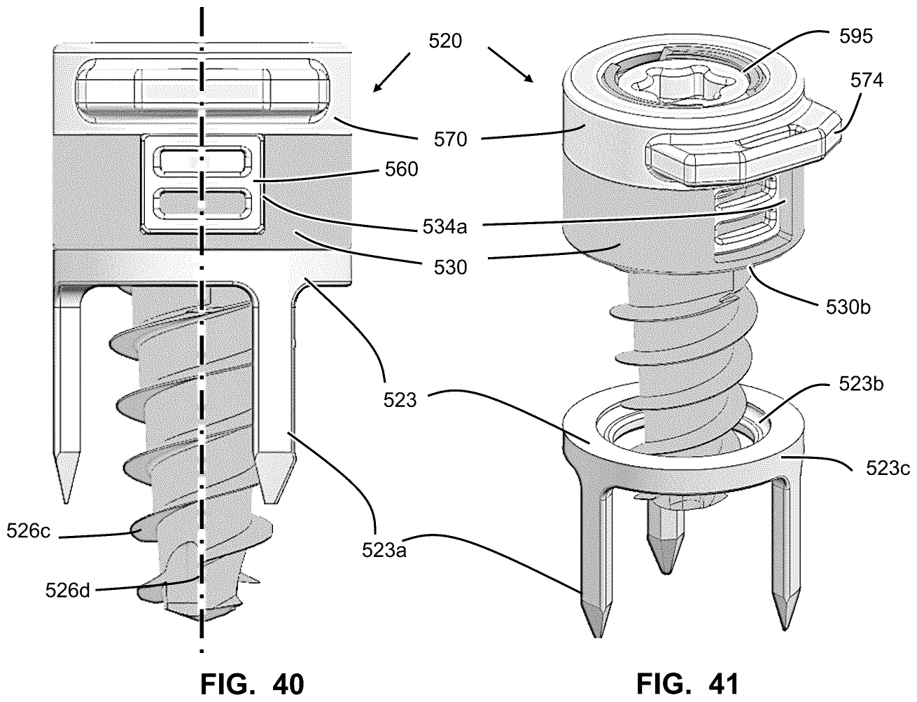

FIGS. 40 and 41 depict an anchoring assembly 520 according to another embodiment of the present invention. Anchoring assembly 520 shares some features with anchoring assembly 220 previously shown and discussed (as well as other anchoring assemblies X20 shown herein), except for the use of a staple 523 for securement of assembly 520 to a bone.

FIGS. 40 and 41 show and anchoring assembly 520 that includes a staple 523 having a plurality of bone-penetrating projections 523a. Staple 523 includes a base 523c interconnecting the projections, and also having an inner shoulder 523b which is adapted and configured to loosely receive within it the under surface 530b of head 530. As the threads 526c of anchor body 526 are attached to a bone, the under surface 530b applies a compressive force to recess 523b, and by way of base 523c into the projections 523a. The use of a staple X23 in any of the anchors X20 shown herein can provide for increased stability of the anchor 520 on the bone.

Various aspects of different embodiments of the present invention are expressed in paragraphs Z1 to Z8 as follows:

Z1. One embodiment of the present invention pertains to an apparatus for fixation of a bone. Preferably, the embodiment includes an anchoring member having a head and a body, said body having a length and being externally threaded from the head to the tip, said head and said body defining an internally threaded pocket that extends distally from said head such that the internal threads and the external threads overlap along the length.

Z2. One embodiment of the present invention pertains to an apparatus for fixation of a bone. Preferably, the embodiment includes an anchoring member having a head and a shaft with a tip, said shaft being externally threaded from the head to the tip, said shaft defining an internally threaded pocket that extends from said head toward the tip, the pocket having a bottom proximate to the tip, the pocket being adapted and configured to receive therein a flexible member along a path, said head having an outer surface that includes an entrance and an exit each on opposing sides of said head. Preferably, the embodiment includes a first screw threadably received by the internal threads and adjustable to a range of locations in the pocket encircled by at least some of the external threads. In still further embodiments, the path has a length from the entrance under said set screw and to the exit, the length being variable corresponding to the placement of said set screw within the range of locations.

Z3. One embodiment of the present invention pertains to an apparatus for fixation of a bone. Preferably, the embodiment includes an anchoring member having a head and a shaft with a distal tip, said shaft being externally threaded along an axis, said anchoring member defining an internal pocket that extends distally from said head toward the tip, said head having an outer surface that includes an entrance and an exit each on opposing sides of the outer surface of said head, the pocket including a first groove extending distally and axially from the entrance, the pocket including a second groove extending distally and axially from the exit, the entrance, exit, first groove, and second groove each being adapted and configured to slidingly receive therein a flexible member along a path.

Z4. One embodiment of the present invention pertains to an apparatus for fixation of a bone. Preferably, the embodiment includes an anchoring member having a head and a shaft with a tip, said shaft being externally threaded from the head to the tip, said head including a circumferential groove extending at least partially around the outer surface of said head. Preferably, the embodiment includes a separable ring adapted and configured to fit within the circumferential groove. Preferably, the embodiment includes an arm having a first end pivotally coupled to one of said head or said ring and a second free end, the second free end being adapted and configured to releasably lock to the other of said head or said ring.

Z5. One embodiment of the present invention pertains to an apparatus for fixation of a bone. Preferably, the embodiment includes a head including a circumferential groove extending at least partially around the outer surface of said head, the groove including a first projection extending within the groove. Preferably, the embodiment includes a separable ring adapted and configured to be received within the groove, said first ring including a mooring location adapted and configured for coupling to a flexible member, said ring including a spring member movable between a first free state and a second radially expanded state, the spring member including a second projection extending from said ring. Preferably, the embodiment includes an anchoring member adapted and configured for attachment to a bone, said anchoring member supporting said head relative to the bone. In still further embodiments, rotation of said ring within the groove results in the first projection contacting the second projection and driving the spring member to the second expanded state.

Z6. One embodiment of the present invention pertains to an apparatus for fixation of a bone. Preferably, the embodiment includes a head adapted and configured for implantation in an animal and including a first groove extending at least partially around the outer surface of said head, said head having a top. Preferably, the embodiment includes a first separable ring having an inner surface, said first ring adapted and configured to fit over the top of said head, said first ring including a mooring location adapted and configured for attachment to an end of a length of a flexible member, said first ring including a second groove extending at least partially around the inner surface of said first ring. Preferably, the embodiment includes a second separable split ring adapted and configured to fit within at least a portion of the first groove and within at least a portion of the second groove. Preferably, the embodiment includes means for anchoring an implanted head to a bone.

Z7. One embodiment of the present invention pertains to an apparatus for fixation of a bone. Preferably, the embodiment includes an anchoring member having a head and a shaft with a tip, said shaft being externally threaded from the head to the tip, said head including a circumferential groove extending at least partially around the outer surface of said head, said head including a fastening feature. Preferably, the embodiment includes a separable ring adapted and configured to fit within the circumferential groove, said ring including a first mooring location adapted and configured for attachment to an end of a length of a flexible member. Preferably, the embodiment includes a separable member adapted and configured to be retained in position by the fastening feature, said member including a second mooring location adapted and configured for attachment to an end of a length of flexible member.

Z8. One embodiment of the present invention pertains to an apparatus for fixation of a bone. Preferably, the embodiment includes an anchoring member having a head and a shaft with a distal end, said shaft being externally threaded with threads adapted and configured for attachment to a bone, said head having an entrance and an exit each adapted and configured for sliding contact with a flexible member and each leading to an internal pocket that extends from said head toward the distal end, the pocket being adapted and configured to receive therein a portion of the flexible member.

Preferably, the embodiment includes a set screw threadably receivable by said head, said set screw including a through bore permitting access to the internal pocket when said set screw is received by said head.

Yet other embodiments pertain to any of the previous statements Z1 to Z8, which are combined with one or more of the following other aspects. It is also understood that any of the aforementioned Z paragraphs include listings of individual features that can be combined with individual features of other Z paragraphs.

Wherein said shaft having an axis and being externally threaded from the head to the tip, said head and said shaft defining an internally threaded pocket that extends from said head toward the tip such that the internal threads and the external threads overlap along a length of the axis, the external threads and internal threads being coaxial along the axis of said shaft.

Wherein said shaft defining an internally threaded pocket that extends from said head toward the tip, the pocket having a bottom proximate to the tip, the pocket being adapted and configured to receive therein a flexible member along a path, said head having an outer surface that includes an entrance and an exit each on opposing sides of the outer surface of said head, the pocket including a first groove extending between the entrance and the bottom, the pocket including a second groove extending between the exit and the bottom.

Which further comprises a set screw threadably received by the internal threads and adjustable to a range of locations in the pocket encircled by the external threads.

Wherein the path has a length from the entrance through the first groove under said set screw through the second groove and to the exit, the length being variable corresponding to the placement of said set screw within the range of locations.

Which further comprises a separable ring adapted and configured to be received within the groove, said first ring including a mooring location adapted and configured for coupling to a flexible member, said ring including a spring member movable between a first free state and a second radially expanded state, the spring member including a second projection extending radially inwardly from the inner diameter of said ring.

Which further comprises a first separable ring having an inner surface, said first ring adapted and configured to fit over the top of said head, said first ring including a mooring location adapted and configured for attachment to an end of a length of a flexible member, said first ring including a second circumferential groove extending at least partially around the inner surface of said first ring; and a second separable split ring adapted and configured to fit between the first circumferential groove and the second circumferential.

Which further comprises a separable member adapted and configured to be retained in position by the fastening feature, said member including a second mooring location adapted and configured for attachment to an end of a length of flexible member.

Which further comprises a separable compression pad placed between the bottom of said set screw and at least one of the entrance or ext.

Wherein a set screw threadably receivable by said head, said set screw including a through bore in general alignment with the internal pocket when said set screw is received by said head.

Wherein the external threads are tapered.

Which further comprises a set screw having opposing proximal and distal faces and external threads between the proximal and distal faces compatible with the internal threads, the set screw having a length between the faces, the length being less than the overlap.

Which further comprises a set screw having opposing proximal and distal faces and second external threads between the proximal and distal faces compatible with the internal threads, and a locking screw having opposing top and bottom faces and third external threads between the top and bottom faces compatible with the internal threads, wherein said set screw and said locking screw are each threadably received within the internally threaded pocket.

Which further comprises a first separate screw received within the internally threaded pocket, and a second separate screw received within the internally threaded pocket, said first separate screw being placed distally from said second separate screw.

Wherein said head includes an entrance adapted and configured to receive therein a flexible member and an exit adapted and configured to receive therein the flexible member, the entrance and exit being on opposing sides of said head, the pocket including a first groove extending from the entrance toward the tip and through the overlapping length, the pocket including a second groove extending from the exit toward the tip and through the overlapping length, the first groove and the second groove being unthreaded.

Wherein said head has a perimeter surface above the shaft, at least a portion of the perimeter surface defining one or more sections of a circumferential groove, the one or more sections of the circumferential groove being provided on opposing sides of said head.

Which further comprises a separable ring adapted and configured to fit within the circumferential groove

Wherein said head has a perimeter surface above the shaft, at least a portion of the perimeter surface defining one or more sections of a perimeter groove, and which further comprises a separable ring adapted and configured to fit within the perimeter groove

Wherein the one or more sections of the groove being provided on opposing sides of said head.

Wherein the internal threads are straight threads.

Which further comprises a pad having an underside adapted and configured for sliding contact with the flexible member, said first screw having a top surface and a bottom surface, said pad being located in contact with the bottom surface.

Wherein which further comprises a second screw threadably received by the internal thread and adapted and configured to place a compressive force against said first screw.

Wherein said head has a perimeter surface above the shaft, at least a portion of the perimeter surface defining one or more sections of a groove, the one or more sections of the groove being provided on opposing sides of said head.

Wherein said head includes a circumferential groove, and which further comprises a separable ring adapted and configured to be received within the circumferential groove.

Wherein the external threads are tapered and the internal threads are straight threads.

Which further comprises a pad having an underside adapted and configured for sliding contact with the flexible member, and a set screw having a bottom surface, said pad being located in contact with the bottom surface.

Which further comprises a pad having first and second lateral sides, and an underside adapted and configured for sliding contact with the flexible member, wherein the first lateral side of said pad is slidingly received in the first groove and the second lateral side of said pad is slidingly received in the second groove.

Which further comprises a set screw adapted and configured to be threadably received by the internal thread between the first groove and the second groove.

Wherein the first projection extends radially outwardly from the groove

Wherein the second projection extends radially inwardly from the inner diameter of said ring.

Wherein the first projection extends outwardly from the groove and the second projection extends inwardly from the inner diameter of said ring.

Wherein after the spring member is in the second expanded state further rotation of the ring in the same direction within the groove results in the spring member returning to the first free state.

Wherein rotation of the free state spring member in an opposite direction within the groove results in abutment of the first projection with the second project preventing further rotation in that opposite direction.

Wherein the second projection includes a ramping surface adapted and configured to move the spring member to the expanded state during sliding contact with the first projection.

Wherein said ring includes a third projection extending radially inwardly from the inner diameter of said ring, wherein the third projection abuts with a wall of the groove to block removal of said ring from said head.

Wherein the first groove is a circumferential groove and the second groove is a second circumferential groove.

Wherein said outer surface includes a tapered surface adapted and configured to receive thereon said second ring.

Wherein said inner surface includes a tapered surface adapted and configured to receive therein said second ring.

Wherein at least a portion of the outer surface is conical.

Wherein said second ring includes a gap in the circumference, said second ring has a first free state in which the outer diameter of said second ring fits within the first groove, and a second compressed state in which said second ring fits within the second groove.

Which further comprises a length of flexible tether having two ends, with one end attached to the first mooring location and the other end attached to the second mooring location.

Which further comprises a first length of flexible tether having an end attached to the first mooring location and a second length of flexible tether having an end attached to the second mooring location.

Wherein the fastening feature has a first shape and said separable member has a second shape adapted and configured to align with the first shape for retention of said separable member.

Wherein the second shape is the shape of the perimeter of said separable member, and the first shape includes the shape of a recess in said head.

Wherein the fastening feature is a recess having a first shape in the outer surface of said head, said separable member has a second shape that is complementary to said first shape.

Wherein the second shape is the shape of the perimeter of said separable member, and the first shape is sized to permit placement of said separable member within the recess.

Wherein tightening of said set screw in said head results in compression of the flexible member between said set screw and said head.

Wherein the compression is between the bottom of said screw and at least one of the entrance or exit.

Wherein the compression is between the side of said screw and the internal pocket.

Wherein the compression is between the side of said screw and at least one of the entrance or exit.

Wherein said shaft has a first axis of symmetry, said set screw has a second axis of symmetry, and the first and second axes are parallel.

Which further comprises a tool having a handle and an end, the size and shape of the end being adapted and configured to be received through the through bore and into the internal pocket.

While the inventions have been illustrated and described in detail in the drawings and foregoing description, the same is to be considered as illustrative and not restrictive in character, it being understood that only certain embodiments have been shown and described and that all changes and modifications that come within the spirit of the invention are desired to be protected.

1. An implantable apparatus for fixation of a bone, comprising:
an implantable anchoring member having a head and a shaft with a closed tip, said shaft having an axis and being externally threaded from the head to the tip, said head and said shaft defining an internally threaded pocket that extends from said head toward the tip such that the internal threads and the external threads overlap along a length of the axis, the external threads and internal threads being coaxial along the axis of said shaft,
wherein said internally threaded pocket is adapted and configured to slidably house a portion of a tether.

2. The apparatus of claim 1, wherein the external threads are tapered.

3. The apparatus of claim 1, further comprising:
a first separate screw received within the internally threaded pocket; and
a second separate screw received within the internally threaded pocket, said first separate screw being placed distally from said second separate screw.

4. The apparatus of claim 1, further comprising:
a set screw having opposing proximal and distal faces and second external threads between the proximal and distal faces compatible with the internal threads; and
a locking screw having opposing top and bottom faces and third external threads between the top and bottom faces compatible with the internal threads,
wherein said set screw and said locking screw are each threadably received within the internally threaded pocket.

5. The apparatus of claim 1, further comprising:
a set screw having opposing proximal and distal faces and external threads between the proximal and distal faces compatible with the internal threads, the set screw having a length between the faces, the length being less than the overlap.

6. The apparatus of claim 1, wherein said head includes an entrance adapted and configured to receive therein a flexible member and an exit adapted and configured to receive therein the flexible member, the entrance and exit being on opposing sides of said head, the pocket including a first groove extending from the entrance toward the tip and through the overlapping length, the pocket including a second groove extending from the exit toward the tip and through the overlapping length, the first groove and the second groove being unthreaded.

7. The apparatus of claim 1, wherein said head has a perimeter surface above the shaft, at least a portion of the perimeter surface defining one or more sections of a circumferential groove.

8. The apparatus of claim 7, further comprising:
a separable ring adapted and configured to fit within the circumferential groove.

9. An implantable apparatus for fixation of a bone, comprising:
an implantable anchoring member having a head and a shaft with a tip, said shaft being externally threaded from the head to the tip, said shaft defining an internally threaded pocket that extends from said head toward the tip, the pocket having a bottom proximate to the tip, the pocket being adapted and configured to receive therein a flexible member along a path, said head having an outer surface that includes an entrance and an exit each on opposing sides of said head; and
a first screw threadably received by the internal threads and adjustable to a range of locations in the pocket encircled by at least some of the external threads; threads,
wherein the path has a length from the entrance, under said first screw and across said bottom of said pocket, and to the exit, the length being variable corresponding to the placement of said first screw within the range of locations.

10. The apparatus of claim 9, further comprising:
a separable ring,
wherein said head has a perimeter surface above the shaft, at least a portion of the perimeter surface defines one or more sections of a perimeter groove, and said separable ring is adapted and configured to fit within the perimeter groove.

11. (canceled)

12. The apparatus of claim 9, further comprising:
a pad having an underside adapted and configured for sliding contact with the flexible member, said first screw having a top surface and a bottom surface, said pad being located in contact with the bottom surface.

13. The apparatus of claim 9, further comprising:
a second screw threadably received by the internal thread and adapted and configured to place a compressive force against said first screw.

14. The apparatus of claim 9, wherein the internal threads are straight threads.

15. An implantable apparatus for fixation of a bone, comprising:
an implantable anchoring member having a head and a shaft with a distal tip, said shaft being externally threaded along an axis, said anchoring member defining an internal pocket that extends distally from said head toward the tip, said head having an outer surface that includes an entrance and an exit each on opposing sides of the outer surface of said head, the pocket including a first groove extending distally and axially from the entrance, the pocket including a second groove extending distally and axially from the exit, the first groove and the second groove being angularly spaced apart around said axis, with the entrance, exit, first groove, and second groove each being adapted and configured to slidably retain therein respective portions of a flexible member along a path.

16. The apparatus of claim 15, wherein said head has a perimeter surface above the shaft, at least a portion of the perimeter surface defining one or more sections of a groove.

17. The apparatus of claim 15, further comprising:
a separable ring,
wherein said head includes a circumferential groove, and said separable ring is adapted and configured to be received within the circumferential groove.

18. The apparatus of claim 15 wherein the internal pocket of said shaft is an internally threaded pocket, the external threads are tapered, and the internal threads are straight threads.

19. The apparatus of claim 15, further comprising:
a pad having an underside adapted and configured for sliding contact with the flexible member; and
a set screw having a bottom surface,
wherein said pad is located in contact with the bottom surface.

20. The apparatus of claim 15, further comprising:
a pad having first and second lateral sides and having an underside adapted and configured for sliding contact with the flexible member,
wherein the first lateral side of said pad is slidably positioned in the first groove and the second lateral side of said pad is slidably positioned in the second groove.

21. The apparatus of claim 15, further comprising:
a set screw adapted and configured to be threadably received by the internal thread between the first groove and the second groove,
wherein the internal pocket of said shaft is an internally threaded pocket.

22-78. (canceled)

79. The apparatus of claim 9, wherein the path within the pocket for the flexible member is not threaded.

80. The apparatus of claim 15, wherein the path within the pocket for the flexible member is not threaded.
